(19) United States
(12) Patent Application Publication
(10) Pub. No.: US 2025/0090130 A1
(43) Pub. Date: Mar. 20, 2025

(54) CO-REGISTRATION TECHNIQUES BETWEEN COMPUTED TOMOGRAPHY IMAGING SYSTEMS AND HISTOTRIPSY ROBOTIC SYSTEMS

(71) Applicant: HistoSonics, Inc., Plymouth, MN (US)

(72) Inventors: Jon D. SCHELL, Shoreview, MN (US); John SNELL, Charlottesville, VA (US); Matthew A. ROOTES, Plymouth, MN (US); Daniel RAUSCH, Plymouth, MN (US); Joshua STOPEK, Minneapolis, MN (US)

(21) Appl. No.: 18/890,580

(22) Filed: Sep. 19, 2024

Related U.S. Application Data
(60) Provisional application No. 63/583,686, filed on Sep. 19, 2023.
(60) Provisional application No. 63/662,235, filed on Jun. 20, 2024.

(51) Int. Cl. A61B 6/58; A61B 6/40

(52) U.S. Cl. CPC A61B 6/583; A61B 6/4085

(57) ABSTRACT

Histotripsy therapy systems and methods configured for the treatment of tissue are provided, which may include any number of features. Systems and methods for registering a coordinate system of a cone-beam computed tomography (CT) imaging device and a robotic histotripsy system are also provided, including robotically driving a phantom connected to a robotic arm to an imaging location, importing cone-beam CT images captured by a cone-beam CT imaging device of the phantom, displaying an image from the imported cone-beam CT images of the phantom, wherein the displayed image depicts at least one marker in the phantom, assessing a number and orientation of the at least one marker in the imported cone-beam CT images compared to a known number and orientation of the at least one marker in the phantom, and registering a coordinate system of the cone-beam CT imaging device to a coordinate system of the histotripsy system.

### PRIORITY CLAIM

This patent application claims priority to U.S. Provisional Application No. 63/583,686, titled “HISTOTRIPSY SYSTEMS AND METHODS UTILIZING CO-REGISTRATION BETWEEN CT IMAGING AND ROBOTIC SYSTEM” and filed on Sep. 19, 2023, and also claims priority to U.S. Provisional Application No. 63/662,235, titled “CONE-BEAMED COMPUTED TOMOGRAPHY HISTOTRIPSY SYSTEMS AND METHODS” and filed on Jun. 20, 2024, each of which are herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure is directed to high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect is directed to a method of registering a coordinate system of a cone-beam computed tomography (CT) imaging device and a robotic histotripsy system. The method includes robotically driving a phantom connected to a robotic arm to an imaging location; importing cone-beam CT images captured by a cone-beam CT imaging device of the phantom; displaying an image from the imported cone-beam CT images of the phantom, where the displayed image depicts at least one marker in the phantom; assessing a number and orientation of the at least one marker in the imported cone-beam CT images compared to a known number and orientation of the at least one marker in the phantom; and registering a coordinate system of the cone-beam CT imaging device to a coordinate system of the histotripsy system. Other embodiments of this aspect include corresponding computer systems, apparatus, and computer programs recorded on one or more computer storage devices, each configured to perform the actions of the methods.

Implementations may include one or more of the following features. The method may include: driving the phantom to an iso-center of the cone-beam CT imaging device. The method may include: driving the phantom to a non-iso-center location of the cone-beam CT imaging device. The method may include: driving the phantom to a pre-determined location of the cone-beam CT imaging device. The method may include: aligning a light alignment feature of the cone-beam CT imaging device with an iso-center alignment marker on the phantom. The method may include: adjusting a position of the phantom to a center of the cone-beam CT imaging device field of view, prior to capturing images of the phantom. A light alignment feature of the cone-beam CT imaging device is aligned with one or more crosshairs on the phantom. The method may include: attaching the phantom to the robotic arm of the histotripsy system. The method may include: arranging the cone-beam CT imaging device and the histotripsy system perpendicular to a patient bed. The method may include: locking wheels of the cone-beam CT imaging device and the histotripsy system. The method may include: confirming clearance of the cone-beam CT imaging device and the robotic arm of the histotripsy system. The method may include confirming a quality of the cone-beam CT images prior to importing the cone-beam CT images. Implementations of the described techniques may include hardware, a method or process, or computer software on a computer-accessible medium.

Another general aspect of the disclosure is a system for registration. The system includes a robotic arm operably connected to a histotripsy system; a phantom operable connected to the robotic arm; and a computing device operably connected to histotripsy system, the computing device including a processor and a memory, the memory storing therein instructions that when executed by the processor cause the computing device to perform steps of: driving the robotic arm to an imaging location; imports cone-beam computed tomography (CT) images of the phantom; determines a number and orientation of markers within at least one of the imported cone-beam CT images; and registers a coordinate system of the histotripsy system with a coordinate system of a cone-beam CT imaging device. Other embodiments of this aspect include corresponding computer systems, apparatus, and computer programs recorded on one or more computer storage devices, each configured to perform the actions of the methods.

Implementations may include one or more of the following features. The system where the instructions when executed by the processor cause the computing device to display instruction via a user-interface to drive the phantom to an iso-center of the cone-beam CT imaging device. The instruction when executed by the processor causes the computing device to display instructions via a user interface to align a light alignment feature of the cone-beam CT imaging device with an iso-center alignment feature on the phantom. The instructions when executed by the processor cause the computing device to display instructions via a user interface to adjust a position of the phantom to a center of the cone-beam CT imaging device field of view, prior to capturing images of the phantom. The instructions when executed by the processor cause the computing device to display instructions via the user interface to align a light alignment feature of the cone-beam CT imaging device with one or more crosshairs on the phantom. The instructions when executed by the processor cause the computing device to display instructions via a user interface to attach the phantom to the robotic arm. The instructions when executed by the processor cause the computing device to display instructions via the user interface to lock wheels of the cone-beam CT imaging device and the histotripsy system. The instructions when executed by the processor cause the computing device to display instructions via a user interface to arrange the cone-beam CT imaging device and the histotripsy system perpendicular to a patient bed. The instructions when executed by the processor cause the computing device to display instructions via a user interface to confirm clearance of the cone-beam CT imaging device and the robotic arm. The instructions when executed by the processor cause the computing device to display instructions via a user interface to confirm a quality of the cone-beam CT images prior to importing the cone-beam CT images. The method may include: adjusting a position of the phantom to a center of the cone-beam CT imaging device field of view, prior to capturing images of the phantom. A light alignment feature of the cone-beam CT imaging device is aligned with one or more crosshairs on the phantom. The method may include: driving the phantom to a pre-determined location of the cone-beam CT imaging device. Implementations of the described techniques may include hardware, a method or process, or computer software on a computer-accessible medium.

A further aspect of the disclosure is a method of registering a coordinate system of a cone-beam computed tomography (CT) imaging device and a robotic histotripsy system. The method includes robotically driving a phantom connected to a robotic arm to an imaging location; importing cone-beam CT images captured by a cone-beam CT imaging device of the phantom; displaying an image from the imported cone-beam CT images of the phantom, where the displayed image depicts at least one marker in the phantom; assessing a number and orientation of the at least one marker in the imported cone-beam CT images compared to a known number and orientation of the at least one marker in the phantom; registering a coordinate system of the cone-beam CT imaging device to a coordinate system of the histotripsy system; detecting a location of an optical marker on each of the phantom, the cone-beam CT imaging device and the histotripsy system; and updating the registration of the cone-beam CT imaging device to the histotripsy system based on detected movement of the optical makers on the cone-beam CT imaging device and the histotripsy system. Other embodiments of this aspect include corresponding computer systems, apparatus, and computer programs recorded on one or more computer storage devices, each configured to perform the actions of the methods.

Implementations may include one or more of the following features. The method may include: driving the phantom to an iso-center of the cone-beam CT imaging device. The method may include: driving the phantom to a non-iso-center location of the cone-beam CT imaging device. The method may include: aligning a light alignment feature of the cone-beam CT imaging device with an iso-center alignment marker on the phantom. The method may include: attaching the phantom to the robotic arm of the histotripsy system. The method may include: locking wheels of the cone-beam CT imaging device and the histotripsy system. The method may include: arranging the cone-beam CT imaging device and the histotripsy system perpendicular to a patient bed. The method may include: confirming clearance of the cone-beam CT imaging device and the robotic arm of the histotripsy system. The method may include confirming a quality of the cone-beam CT images prior to importing the cone-beam CT images. Implementations of the described techniques may include hardware, a method or process, or computer software on a computer-accessible medium.

Yet a further aspect of the disclosure is a registration phantom. The registration phantom includes a body; a plurality of markers secured to the body, an angled attachment platform connected to the body, and a connector configured for connection to a robotic arm.

Implementations may include one or more of the following features. The registration phantom where the plurality of markers is arranged in a helix around the body. The registration phantom where crosshairs are used for alignment of the body with an imaging device. The imaging device is a cone-beam computed tomography system. Cone-beam tomography system is registered to a robotic arm of a histotripsy system. The angled attachment platform is connected to the body at an angle selected to avoid robotic arm singularities. The registration phantom may include an iso-center marker. The plurality of markers includes markers of two or more diameters. The registration phantom may include one or more optical markers. The body is cylindrical.

### BRIEF DESCRIPTION OF THE DRAWINGS

The features of the disclosure are set forth with particularity in the claims that follow. A better understanding of the features and advantages of the disclosure will be obtained by reference to the following detailed description that sets forth illustrative embodiments, in which the principles of the disclosure are utilized, and the accompanying drawings of which:

FIG. 1A is a perspective view of a histotripsy system in accordance with the disclosure;

FIG. 1B depicts a therapy transducer and imaging transducer in accordance with the disclosure;

FIG. 2 depicts a histotripsy system deployed to treat a patient in accordance with the disclosure;

FIG. 3 is a plot of a pressure wave during application of a histotripsy ultrasound pulse in accordance with the disclosure;

FIGS. 4A-4D depict a phantom for registration of a histotripsy system with a cone-beam CT imaging device in accordance with the disclosure;

FIGS. 4E-4G illustrate methods for registering an imaging system coordinate system to a robotic positioning system coordinate system.

FIG. 5 is a user interface of an application employed in register a histotripsy system with a cone-beam CT imaging device in accordance with the disclosure;

FIG. 6 is a flow chart depicting a method of registering a cone-beam CT imaging device with a histotripsy system in accordance with the disclosure;

FIG. 7 is a user interface of an application employed in register a histotripsy system with a cone-beam CT imaging device in accordance with the disclosure;

FIG. 8 is a user interface of an application employed in register a histotripsy system with a cone-beam CT imaging device in accordance with the disclosure;

FIG. 9 is a user interface of an application employed in register a histotripsy system with a cone-beam CT imaging device in accordance with the disclosure;

FIG. 10 is a user interface of an application employed in register a histotripsy system with a cone-beam CT imaging device in accordance with the disclosure;

FIG. 11 is a user interface of an application employed in register a histotripsy system with a cone-beam CT imaging device in accordance with the disclosure;

FIG. 12 is a user interface of an application employed in register a histotripsy system with a cone-beam CT imaging device in accordance with the disclosure;

FIG. 13 is a user interface of an application employed in register a histotripsy system with a cone-beam CT imaging device in accordance with the disclosure;

FIG. 14 is a user interface of an application employed in register a histotripsy system with a cone-beam CT imaging device in accordance with the disclosure;

FIG. 15 is a user interface of an application employed in register a histotripsy system with a cone-beam CT imaging device in accordance with the disclosure;

FIG. 16 is a user interface of an application employed in register a histotripsy system with a cone-beam CT imaging device in accordance with the disclosure;

FIG. 17 is a user interface of an application employed in register a histotripsy system with a cone-beam CT imaging device in accordance with the disclosure;

FIG. 18 is a user interface of an application employed in register a histotripsy system with a cone-beam CT imaging device in accordance with the disclosure;

FIG. 19 is a user interface of an application employed in register a histotripsy system with a cone-beam CT imaging device in accordance with the disclosure;

FIG. 20 is a user interface of an application employed to receive and localize target tissue in captured cone-beam CT images in accordance with the disclosure;

FIG. 21 is a flowchart of a method for receiving cone-beam CT images and localize target tissue in accordance with the disclosure;

FIG. 22 is a user interface of an application employed to receive and localize target tissue in captured cone-beam CT images in accordance with the disclosure;

FIG. 23 is a user interface of an application employed to receive and localize target tissue in captured cone-beam CT images in accordance with the disclosure;

FIG. 24 is a user interface of an application employed to receive and localize target tissue in captured cone-beam CT images in accordance with the disclosure;

FIG. 25 is a user interface of an application employed to receive and localize target tissue in captured cone-beam CT images in accordance with the disclosure;

FIG. 26 is a user interface of an application employed to receive and localize target tissue in captured cone-beam CT images in accordance with the disclosure;

FIG. 27 is a user interface of an application employed to receive and localize target tissue in captured cone-beam CT images in accordance with the disclosure;

FIG. 28 is a user interface of an application employed to receive and localize target tissue in captured cone-beam CT images in accordance with the disclosure;

FIG. 29 is a user interface of an application employed to receive and localize target tissue in captured cone-beam CT images in accordance with the disclosure;

FIG. 30 is a user interface of an application employed to receive and localize target tissue in captured cone-beam CT images in accordance with the disclosure;

FIG. 31 is a user interface of an application employed to receive and localize target tissue in captured cone-beam CT images in accordance with the disclosure;

FIG. 32 is a user interface of an application employed to receive and localize target tissue in captured cone-beam CT images in accordance with the disclosure;

FIG. 33 is a user interface of an application employed to receive and localize target tissue in captured cone-beam CT images in accordance with the disclosure;

FIG. 34 is a user interface of an application employed to receive and localize target tissue in captured cone-beam CT images in accordance with the disclosure;

FIG. 35 is a user interface of an application employed to receive and localize target tissue in captured cone-beam CT images in accordance with the disclosure;

FIG. 36 is a user interface of an application employed to receive and localize target tissue in captured cone-beam CT images in accordance with the disclosure;

FIG. 37 is a user interface of an application employed to receive and localize target tissue in captured cone-beam CT images in accordance with the disclosure;

FIG. 38 is a user interface of an application employed to receive and localize target tissue in captured cone-beam CT images in accordance with the disclosure;

FIG. 39 is a user interface of an application employed to receive and localize target tissue in captured cone-beam CT images in accordance with the disclosure;

FIG. 40 is a user interface of an application employed to receive and localize target tissue in captured cone-beam CT images in accordance with the disclosure;

FIG. 41 is a user interface of an application employed to receive and localize target tissue in captured cone-beam CT images in accordance with the disclosure;

FIG. 42 is a user interface of an application employed to plan a treatment using captured cone-beam CT images in accordance with the disclosure;

FIG. 43 is a user interface of an application employed to plan a treatment using captured cone-beam CT images in accordance with the disclosure;

FIG. 44 is a user interface of an application employed to plan a treatment using captured cone-beam CT images in accordance with the disclosure;

FIG. 45 is a user interface of an application employed to plan a treatment using captured cone-beam CT images in accordance with the disclosure; and

FIG. 46 is a user interface of an application employed to plan a treatment using captured cone-beam CT images in accordance with the disclosure.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing, and ablation. Furthermore, due to tissue selective properties, histotripsy may be used to create a cytoskeleton that allows for subsequent tissue regeneration either de novo or through the application of stem cells and other adjuvants. Histotripsy can also be used to cause the release of delivered agents such as chemotherapy and immunotherapy by locally causing the release of these agents by the application of acoustic energy to the targets. As will be described below, the acoustic cavitation system may include various sub-systems, including a cart, therapy, integrated imaging, robotics, coupling and software subsystems. The acoustic cavitation system also may comprise various other components, ancillaries and accessories, including but not limited to computers, processors, memory, software, applications, cables, connectors, networking devices, power supplies, displays, drawers/storage, doors, wheels, and various simulation and training tools, etc. All systems, methods and means creating/controlling/delivering histotripsy are considered to be a part of this disclosure.

### The System

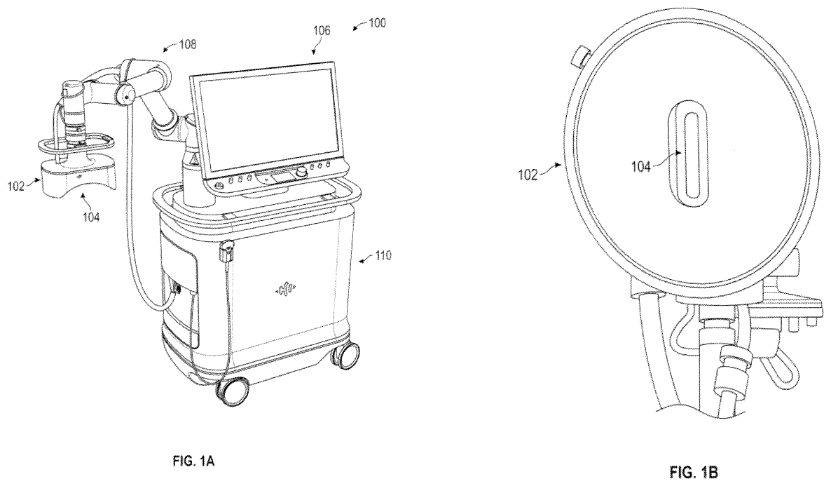

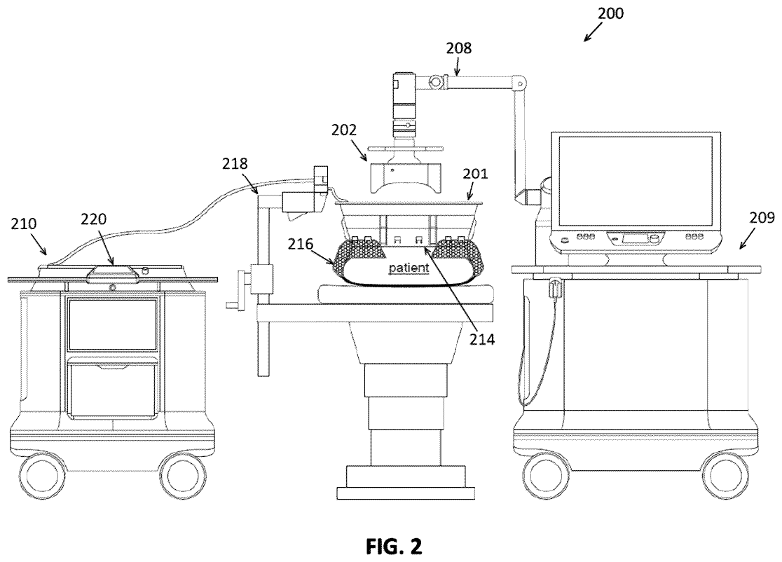

FIG. 1A depicts a histotripsy system 100 in accordance with the disclosure. The histotripsy system 100 includes a therapy transducer 102, an imaging system 104, a display and control panel 106, a robotic positioning arm 108, and a cart 110. The system can further include an ultrasound coupling interface and a source of coupling medium (FIG. 2).

FIG. 1B is a bottom view of the therapy transducer 102 and the imaging system 104. As shown, the imaging system 104 can be positioned in the center of the therapy transducer 102. However, other embodiments can include the imaging system 104 positioned in other locations within the therapy transducer 102, or even directly integrated into the therapy transducer 102. In some embodiments, the imaging system is configured to produce real-time imaging at a focal point of the therapy transducer 102. The system also allows for multiple imaging transducers 104 to be located within the therapy transducer 102 to provide multiple views of the target tissue simultaneously and to integrate these images into a single 3-D image.

The histotripsy system 100 may comprise, and the cart 110 may enclose, one or more of various sub-systems, including a therapy sub-system that can create, apply, focus and deliver acoustic cavitation/histotripsy through one or more therapy transducers 102, an integrated imaging sub-system (or connectivity thereto) allowing real-time visualization and display of the treatment site and histotripsy effect through-out the procedure (e.g., via, a robotics positioning sub-system to mechanically and/or electronically steer the therapy transducer) and further enabled to connect/support or interact with a coupling sub-system to allow acoustic coupling between the therapy transducer 102 and the patient, and software to communicate, control and interface with the system and computer-based control systems (and other external systems) and various other components, ancillaries and accessories, including one or more user interfaces and displays, and related guided work-flows, all working in part or together. The histotripsy system 100 may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described in greater detail below, the cart 110 may be configured and arranged to be used in a radiology environment and in some cases in concert with imaging (e.g., computed tomography (CT), cone beam CT and/or magnetic resonance imaging (MRI) scanning). In other embodiments, the cart 110 may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The cart 110 may also work with a patient surface (e.g., table or bed) to allow the patient to be presented and repositioned in a variety of positions, angles and orientations, including allowing changes to such to be made pre, peri and post-procedurally. The cart 110, and subsystems thereof, may further comprise the ability to interface and communicate with one or more external imaging or image data management and communication systems, not limited to ultrasound, CT, fluoroscopy, cone beam CT, positron emission tomography (PET), PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more carts may be configured to work together. As an example, one cart may comprise a bedside mobile cart equipped with one or more robotic arms enabled with a therapy transducer, and therapy generator/amplifier, etc., while a companion cart working in concert and at a distance of the patient may comprise integrated imaging and a console/display for controlling the robotic and therapy facets, analogous to a surgical robot and master/slave configurations.

FIG. 2 illustrates one embodiment of a histotripsy therapy and imaging system 200, including a coupling assembly 201. As described above, a histotripsy therapy and imaging system can include a therapy transducer 202, an imaging system, a robotic positioning arm 208, and a fluidics cart 210. The robotic positioning arm may be attached to a therapy cart, such as cart 209.

The therapy and/or imaging transducers can be disposed within in the coupling assembly 201 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 201 is supported by a mechanical support arm 218 which can be load bearing in the x-y plane but allow for manual or automated z-axis adjustment. The mechanical support arm can be attached to the floor, the patient table, or the fluidics cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The fluidics cart 210 can include additional features, including a fluid tank 220, a cooling and degassing system, and a programmable control system. The fluidics cart is configured for external loading of the coupling membrane with automated control of fluidic sequences. Further details on the fluidics cart are provided below.

### Histotripsy

Histotripsy is achieved by generating short, high amplitude, focused ultrasound pulses to generate a dense, energetic, “bubble cloud,” capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically affect tissue structure, and in some cases liquefy, suspend, solubilize and/or destruct tissue into sub-cellular components.

Histotripsy can be applied in various forms, including: 1) intrinsic-threshold histotripsy which delivers pulses typically with a 1-2 cycles of high amplitude negative/tensile phase pressure exceeding the intrinsic threshold to generate cavitation in the medium (e.g., ˜24-28 MPa for water-based soft tissue), 2) shock-scattering histotripsy which delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) boiling histotripsy which employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism.”

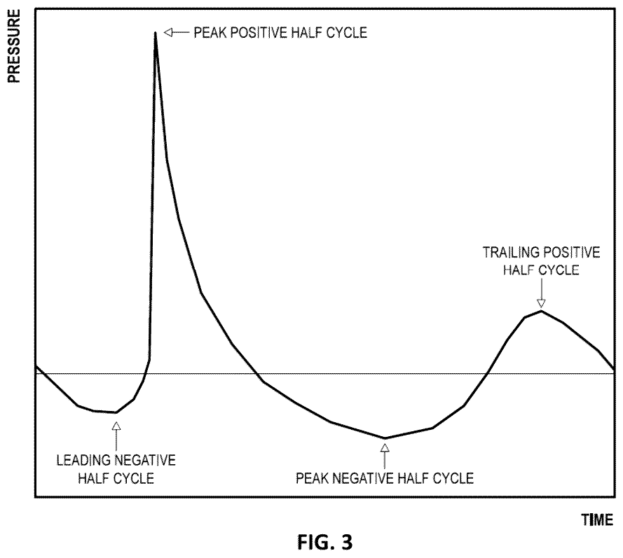

FIG. 3 illustrates an ultrasound pulse that can be used for shock scattering histotripsy. As shown the ultrasound pulse can include a leading negative half cycle, a peak positive half cycle, a peak negative half cycle, and a trailing peak positive half cycle (with the pulse traveling from right to left on the page). As shown, the trailing peak positive cycle has a lower amplitude than the peak positive cycle. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface if the amplitude of those cycles is sufficient, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When the amplitude(s) of positive half cycle(s) of each pulse are limited, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Cone-Beam CT Integration

As described above, in accordance with the disclosure the histotripsy system 100 is integrated with or interoperable with cone-beam CT imaging capabilities including mobile or fixed room systems. Pre-procedural CT, MRI, cone-beam, and even PET images have been previously described in connection with identification of tumors and lesion to receive therapy. In addition, the histotripsy system 100 may include a memory or storage and a combination of firmware and hardware capable of receiving these pre-procedural images so they can be displayed to a user via the display and control panel 106 (e.g., in a user-interface). In addition, in some aspects of the histotripsy system the pre-procedural images can be analyzed and registered to live ultrasound images captured by the imaging system 104 and registered to the ultrasound images and overlaid one another or fused together. By adjusting the transparency (e.g., alpha or color blending, etc.) of the co-registered ultrasound images or the pre-procedural images a composite image is formed allowing the user to better identify the location of a tumor or lesion (or other target tissue) that is difficult to identify in just the ultrasound images. These composite images can be employed, for example, for localizing, targeting, planning of the therapy, real-time monitoring of therapy pr post-treatment assessment and verification. While the use of the pre-procedural images to augment the live ultrasound is effective, improvements are always desirable based on the inherent limitations of ultrasound imaging and/or of end-user experience in interpreting such images.

One challenge with pre-procedural images is that the images are often acquired days and even weeks prior to the actual procedure. Further, they are acquired under very different patient positioning than expressed during a histotripsy procedure. The pre-procedure images may be acquired at full inspiratory breath hold, while during a histotripsy procedure the patient may be normal or controlled tidal volume breathing. This change in breathing pattern results in changes in the position of organs, bones, and other aspects of the patient making registration of the two images sets more challenging.

Further, during acquisition of pre-procedure images no coupling assembly 212 is employed. The addition of the coupling assembly 212, and particularly the coupling medium within the coupling assembly 212 places a significant weight on the patient which can cause deformation and shifting of organs, bones, and other tissues of the patient. While the challenges in using pre-procedure can often be addressed during the histotripsy procedure, particularly where the target tissue can be observed in the live ultrasound images captured by the imaging system 104, challenges remain.

One such challenge is where the target tissue (e.g., a tumor or lesion) which was observable in the preprocedural images cannot be observed in the ultrasound images. This may occur where the target tissue is deeper within the patient than can be observed using ultrasound imaging, where the target tissue is obscured by bone (e.g., ribs, cranium, etc.) or other hard objects (medical devices or medical implants) preventing the ultrasound signals from passing through the relevant tissue beyond the obstruction. In other instances, the healthy tissue and the target tissue may be relative homogonous in its physical properties (e.g., density, elasticity, etc.) resulting in images where it is difficult to discern the target tissue from other tissues. In such instances, the registration must be highly accurate in order for the surgeon to rely on the pre-procedure images for planning of the therapy.

To address these and other challenges, one aspect of the disclosure is directed to the integration and utilization of peri- and intra-procedural cone-beam CT images. The cone-beam CT imaging device is registered to coordinate system of the histotripsy system 100, and particularly to the coordinate system of the robotic positioning arm 108. By registering the cone-beam CT imaging device to the coordinate system of the robotic positioning arm 108 and capturing the intra-procedural cone-beam CT images with the patient positioned for therapy with the coupling assembly 212 placed on the patient for application of the therapy, the forgoing challenges with using preoperative image data are eliminated (e.g., image-to-body deformation, etc.).

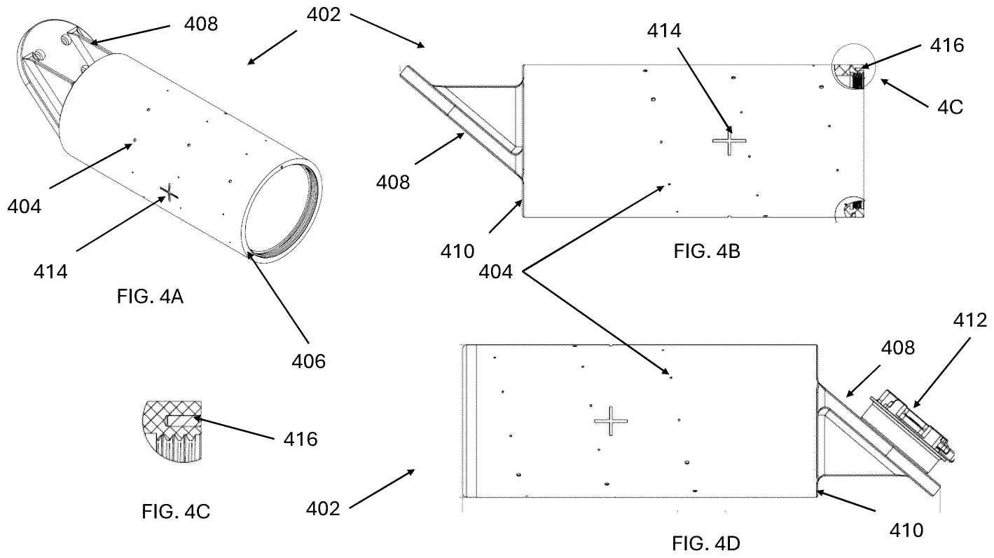

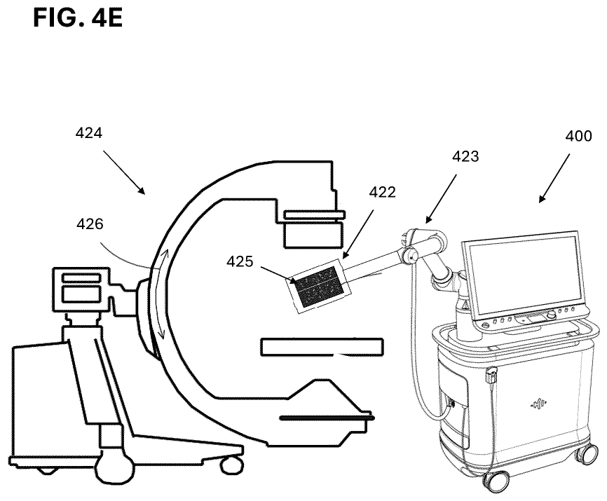

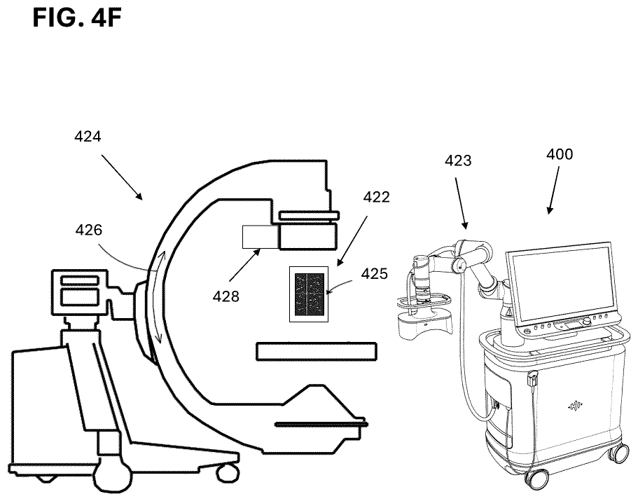

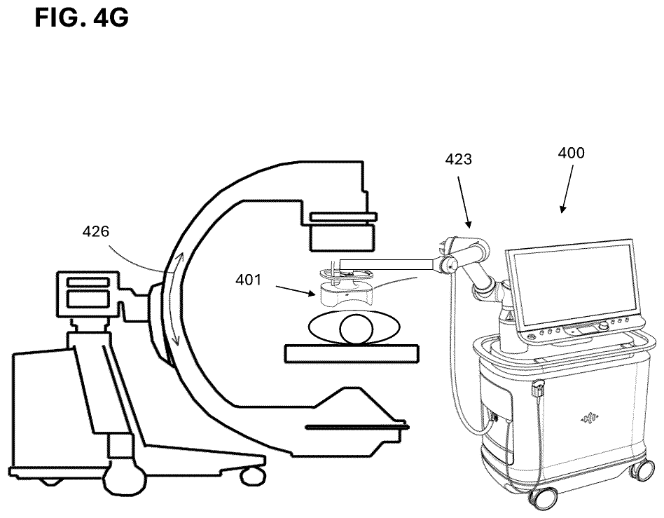

Both the robotic positioning arm 108 and the cone-beam CT imaging device have their own coordinate systems. To enable the ability for patient-specific features (e.g., anatomical locations, structures, tissues, etc.) identified and of interest in the cone-beam CT to be translated to the robotic arm coordinate system, a registration between the cone-beam CT imaging device and the histotripsy system 100 must be performed. This may specifically include a transformation calculation between the cone beam CT imaging coordinate system and the histotripsy or robotic positioning arm 108 coordinate systems, enabling the ability to determine location and orientation of the robot positioning arm 108 (and any end-effectors) in the acquired cone-beam CT images. To register the cone-beam CT imaging device with the robotic positioning arm 108 a phantom 402 (FIG. 4A) is employed. Phantom 402 is configured to mount to an end of the robotic positioning arm 108. The robotic positioning arm 108 is driven to a location where images of the phantom 402 can be acquired by the cone-beam CT imaging device. The histotripsy system 100 is able to determine where in space (e.g., the 3D position and orientation of) the phantom 402 is in accordance with the robotic positioning arm 108 coordinate system. The cone-beam CT imaging device acquires images of the phantom 402. The cone-beam CT imaging device is able to determine its position in the imaging device coordinate system when the images of the phantom 402 were acquired. The phantom 402 includes thereon a number of markers 404. The markers 404 may all be the same size, or they may be of two or more different sizes. The markers 404 are positioned or configured in a known position and orientation from one another, for example in a helix pattern as depicted in FIG. 4A. Because the position of the markers 404 in the phantom in robotic positioning arm 108 coordinates is known by the histotripsy system 100 and the position of the beads in the cone-beam CT imaging coordinates can be determined by the cone-beam CT imaging device, a transformation between the coordinate systems can be determined. Once the transformation is determined, the positions of relevant tissue (e.g., tumors for treatment) identified in the images captured by the cone-beam CT imaging device can be ascertained in the robotic positioning arm 108 coordinate system. With knowledge of the location of the relevant tissue, the robotic positioning arm 108 can be driven to a location (e.g., position and orientation) for application of therapy. In some configurations, this may further the ability to angle and/or rotate around the defined target location to assess or optimize the therapy or access an acoustic window to the target.

The cone-beam CT images are acquired with the patient in the same position and orientation at which therapy will be applied with the coupling assembly 212 in place on the patient. As a result, the location of target tissue for application of therapy identified in the cone-beam CT images can be accurately used for directing the robotic positioning arm 108 to a location to apply the therapy. This positioning of the robotic positioning arm, and particularly the therapy transducer 102 can be achieved with confidence, even in instances where the target cannot be visualized via ultrasound imaging (e.g., via imaging system 104). For example, histotripsy can be employed to apply therapy to targets within or on the brain of a patient and not visible under ultrasound imaging because of the skull of the patient. Indeed, in accordance with the disclosure, no ultrasound imaging is needed to apply therapy when employing peri- or intraprocedural cone-beam CT imaging where the coordinate system of the cone-beam CT imaging device is registered to the coordinate system of the histotripsy system 100. As a result, with the patient in a sedated state there is little to no movement of the target tissue from its location during imaging and the surgeon can proceed with confidence that effective therapy will be applied to the imaged target tissue.

In some embodiments, the histotripsy system 100 may be configured with various features to assess or confirm the registration accuracy. In accordance with these embodiments the alignment of various landmarks (both known or user-defined or selected) that are both visible on cone-beam CT images and ultrasound images can be undertaken. By driving to these positions and confirming that landmark is visible in the ultrasound images at the same location and orientation of the therapy transducer as they appear in the registered cone-beam CT images. This includes for use cases where the target location (e.g., tumor) may only be visible on cone-beam CT. In some examples, if users desire or require assessing the registration, the histotripsy system may allow users to move to one or more defined locations (positions and orientations) and assess the cone-beam CT and ultrasound registration is sufficiently accurate for their procedure. Further, in some embodiments, this may include features that allow users to save robot positions and orientations to allow them to return to said positions and orientations as preferred.

Referring to FIGS. 4A-4D, the phantom 402 is depicted. In accordance with the disclosure the phantom 402 has a generally cylindrical body 406, though other shapes of body 406 are considered within the scope of the disclosure (e.g., rectangular, square, etc.). As shown in FIGS. 4A-4D the plurality of markers 404 are embedded in the body 406 is a helical pattern, though other patterns may be employed without departing from the scope of the disclosure. On a proximal end, extending from the cylindrical body 406 is an attachment platform 408. The attachment platform 408 extends at an angle (e.g., between 30 and 60 degrees) from the end plate 410 of the cylindrical body 406. The angle of the attachment platform enables the robotic positioning arm 108 to achieve a desired position and orientation of the phantom 402 that avoids a lock-out position and singularities of the robotic positioning arm 108. As is known to those of ordinary skill in the art, in certain positions a multi-joint robotic positioning arm 108 can effectively be driven into a lock-out position, which does not allow for further articulation. By incorporating the attachment platform 408 at an angle to the end plate 410, the robotic positioning arm 108 is able to be articulated and driven such that the phantom 402 is in a desired position for imaging (e.g., the center of the imaging field of view of the cone-beam CT imaging device) with respect to a cone-beam CT imaging device without reaching the lock-out position. The attachment platform 408 is configured to receive a connector 412. The connector 412 mates with a corresponding connector on the end of the robotic positioning arm 108.

Formed on the body 406 of the phantom 402 are a variety of alignment features. A first such alignment feature are crosshairs 414. In some examples, the crosshairs 414 may be machined or cut into or painted onto the body 406 of the phantom 402. As described in greater detail below the crosshairs 414 provide a target for alignment tools of the cone-beam CT imaging device. In some cone-beam CT imaging devices include light emitters (e.g., light emitting diodes) which project a concentrated light that can be used to positioning the phantom 402 in a desired position to optimize imaging with the cone-beam CT imaging device. By aligning the crosshairs 414 and the light from the light sources, positioning of the phantom 402 relative to the cone-beam CT imaging device is confirmed prior to acquisition of the images. In some configurations wherein the cone-beam CT may not be configured with laser alignment, the crosshairs may be aligned using orthogonal fluoroscopic views and wherein the fluoroscopic isocenter is aligned to the center of the marker pattern.

In some configuration a further feature of the phantom 402 is an iso-center confirmation marker 416. As described in greater detail below, the iso-center confirmation marker 416 is used post-registration to verify the registration of the coordinate systems of the cone-beam CT imaging device and the robotic positioning arm 108. In this example, the isocenter is visually and fluoroscopically visible and once the isocenter of the imaging volume is determined, a pose is calculated and the robotic positioning arm 108 is driven to move the phantom 402 to place the iso-center confirmation marker 416 to iso-center of the cone-beam CT imaging device. The user may then confirm the location of the iso-center alignment marker relative to the crosshairs and/or fluoroscopic iso-center.

Techniques are provided herein for obtaining the robot coordinate systems for a histotripsy system 400. In some aspects, the techniques require knowing the position of the tool point of the robot arm and the robot pose in base coordinates. In a first embodiment, referring to FIG. 4E, an imaging phantom 422 is attached to the arm of the robotic positioning system 423 in place of the histotripsy treatment head. The imaging phantom can comprise a known pattern of reflectors 425, such as in a helical pattern. The phantom can then the positioned in the imaging space of an imaging system 424 (with the robotic positioning system) and an imaging scan can be performed. In the illustrated embodiment, the imaging system 424 comprises a CT or cone beam CT system that is articulatable along one or more axis 426 to facilitate imaging in multiple imaging planes. The reference geometry of the phantom reflector pattern is then compared to its location in the imaging space. The robot pose (orientation) and location of the phantom in imaging space at the time of the scan allows for calculation of the transformation matrix between the imaging system coordinate system and the robotic positioning system coordinate system.

In a second embodiment, as shown in FIG. 4F, a tracking system 428 is attached to the imaging system 424. Tracking systems may be comprised of optical or electromagnetic tracking systems, as representative examples. The tracking system has a field of view that includes references for the robot tool point and base. Knowing the coordinates of the tool point, base and robot pose at the time of the image acquisition allows for calculation of the transformation matrix between the imaging system coordinate system and the robotic positioning system coordinate system. In some embodiments, the tracking system may be integrated (physically and/or through software), to the imaging system, and may include various sensor locations for tracking objects on or around the patient, patient surface and/or histotripsy system. This can also include the system cart, arm, treatment head, coupling system and/or various components or fixtures on other ancillaries or devices.

Once the registration has been made between the imaging system and the histotripsy system/robotic system, the treatment head 401 can be positioned on the robotic positioning system and a patient can be placed on an operating table in the field of view of the imaging system, as shown in FIG. 4G. Movement of the robotic positioning system, and therefore the treatment head, is now registered with images obtained by the imaging system.

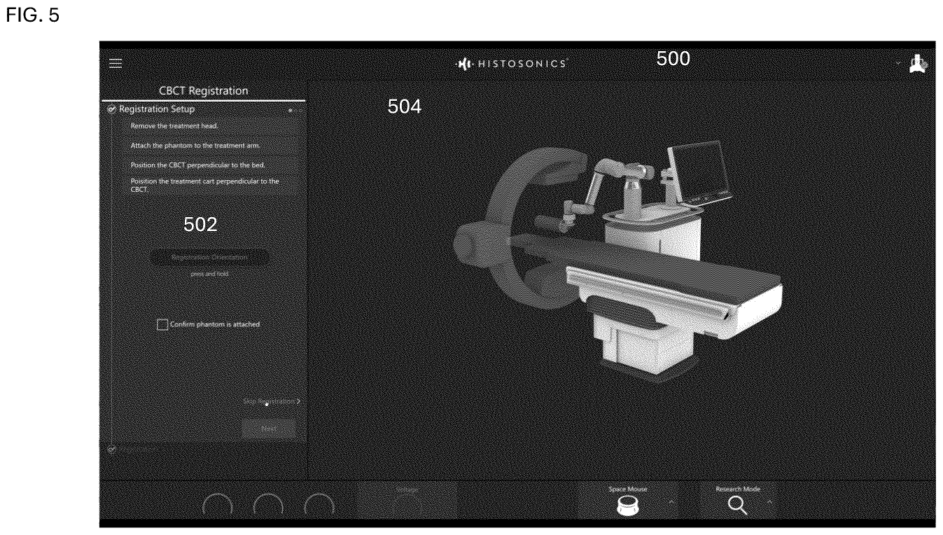

FIG. 5 depicts a user interface 500 for presentation on the display and control panel 106 for a cone-beam CT registration workflow in accordance with the disclosure. The user interface 500 includes an instruction panel 502 detailing steps to be undertaken in connection with the registration workflow. An image panel 504 presents a series of images to assist the user in connection with performing the steps outlined in the instruction panel. The image panel 504 may also present images acquired from the cone-beam CT imaging device, or dropdowns for selection of image data sets, as outlined in greater detail below.

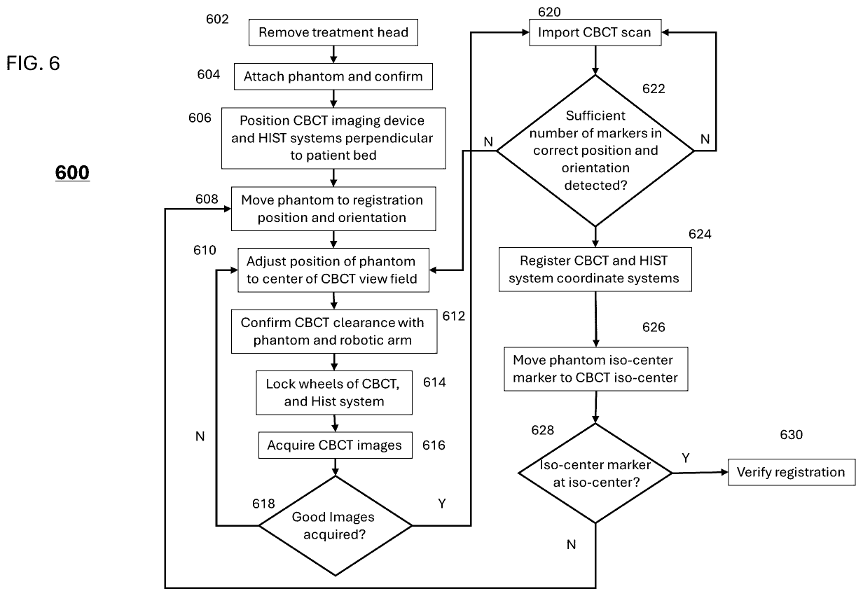

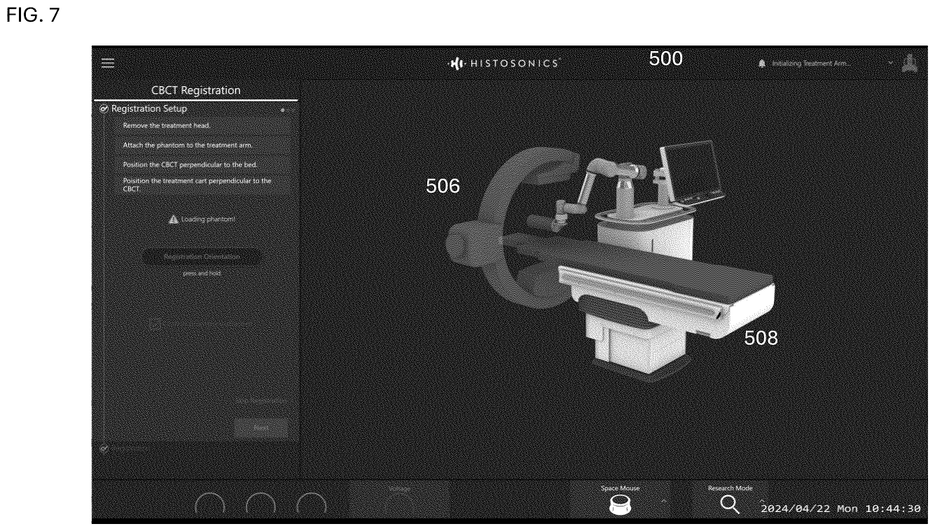

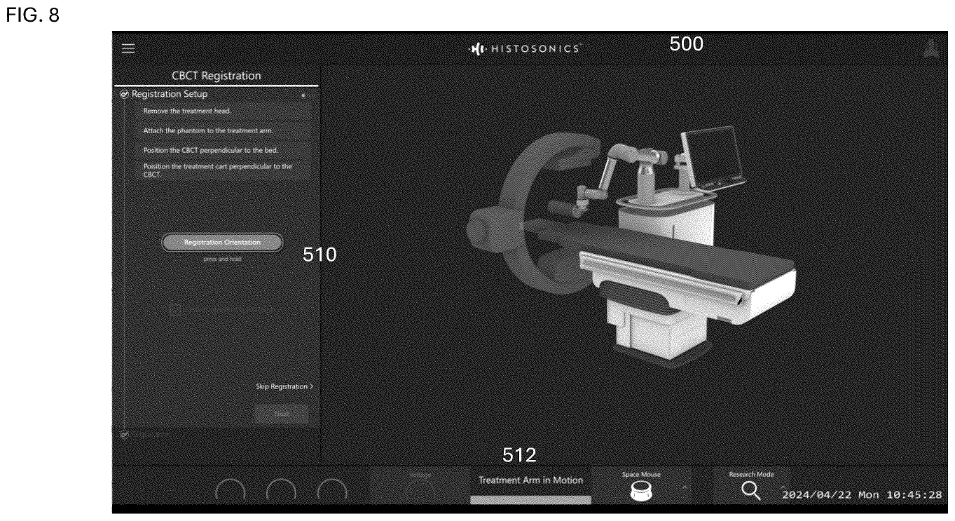

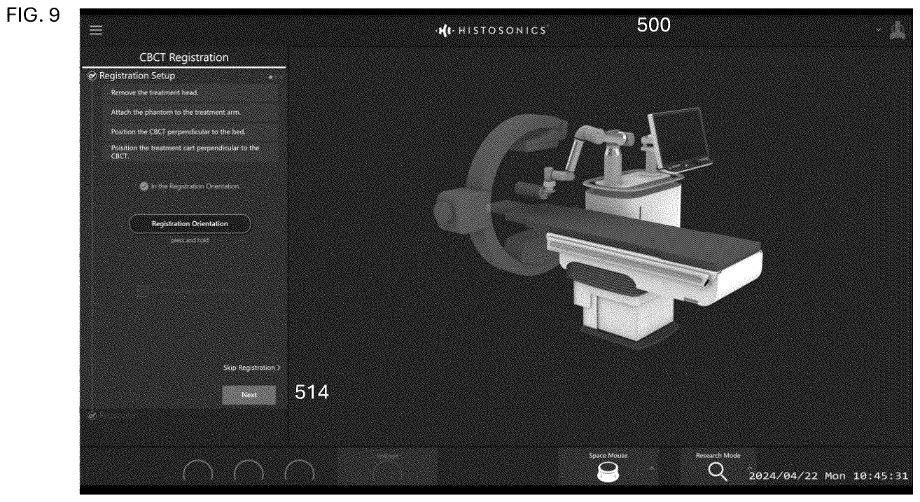

As depicted in FIG. 5, the instruction panel 502 outlines initial steps for the registration of the cone-beam CT imaging device 506 with the robotic positioning arm 108 of the histotripsy system 100, as shown in the image panel 504. As set forth in the instruction panel 502, and as set forth in method 600 in FIG. 6 an initial step of the registration workflow is to remove the treatment head (e.g., the therapy transducer 102) from the robotic positioning arm 108 at step 602. At step 604 as directed in FIG. 7 the phantom 402 is to be attached to the robotic positioning arm 108 and can be confirmed via an input to the user interface 500 (See FIG. 7). At step 606, the cone-beam CT imaging device 506 and the cart (e.g., 210) of the histotripsy system 100 are positioned perpendicular to the patient bed 508. Once the cone-beam CT imaging device 506 and the cart 210 of the histotripsy system 100 are located perpendicular to the bed 508 and the phantom 402 is confirmed as attached, a registration orientation button 510 illuminates and by receiving an input via the display and control panel 106, the robotic positioning arm 108 moves into the registration orientation as noted by movement indicator 512 at step 608 (See FIG. 8). Once the registration orientation is achieved and indicator is depicted on the user interface 500 as shown in FIG. 9 and a next button 514 may be selected.

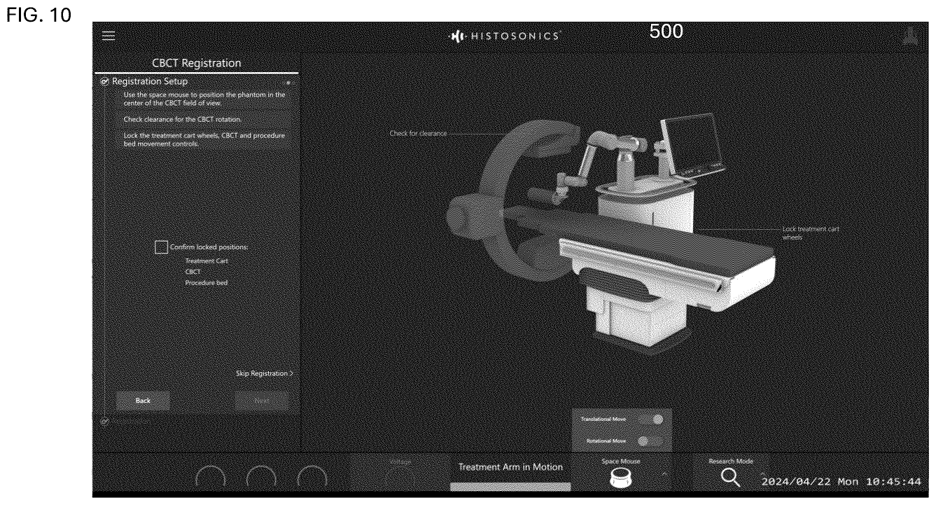

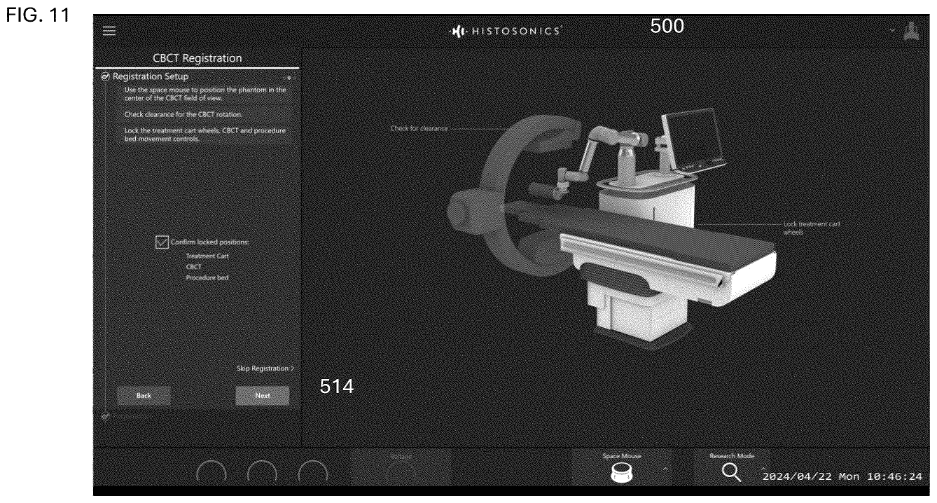

As depicted in FIG. 10, at step 610 the phantom is centered in the cone-beam CT imaging device field of view using, for example, the space mouse operably connected to the display and control panel 106. For example, the light alignment features of the cone-beam CT imaging device 506 can be aligned with the crosshairs 414 on the phantom 402 to confirm the positioning of the phantom 402. At step 612 the clearance of the cone-beam CT imaging device 506 with respect to the phantom 402 and robotic positioning arm 108 is confirmed and at step 614 the wheels of the cone-beam CT imaging device and the histotripsy system 100 are locked in place to prevent movement of the histotripsy system or the cone-beam CT imaging device. Once locked a confirmation can be made via the display and control panel 106 as seen in FIG. 11 and the next button 514 may be selected.

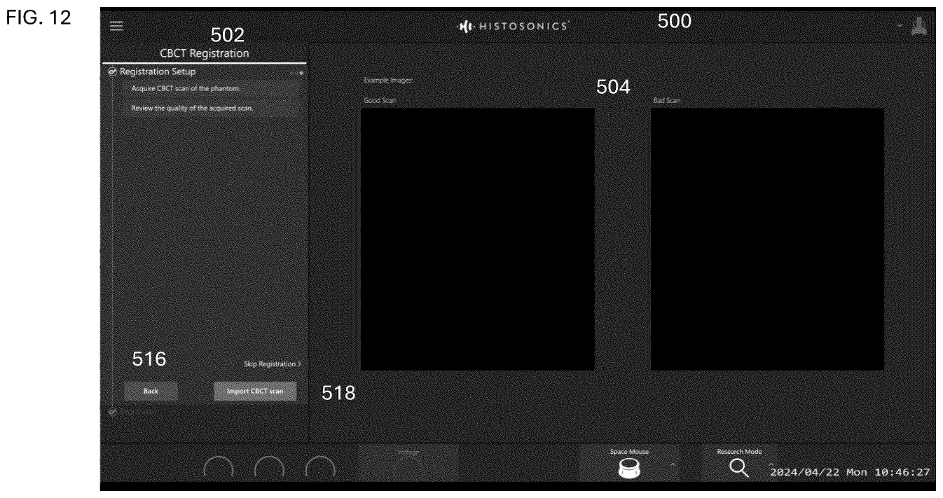

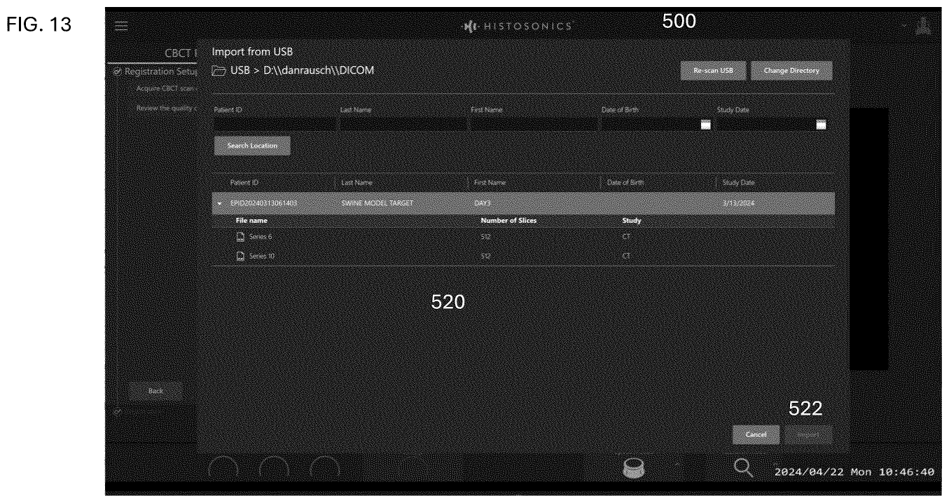

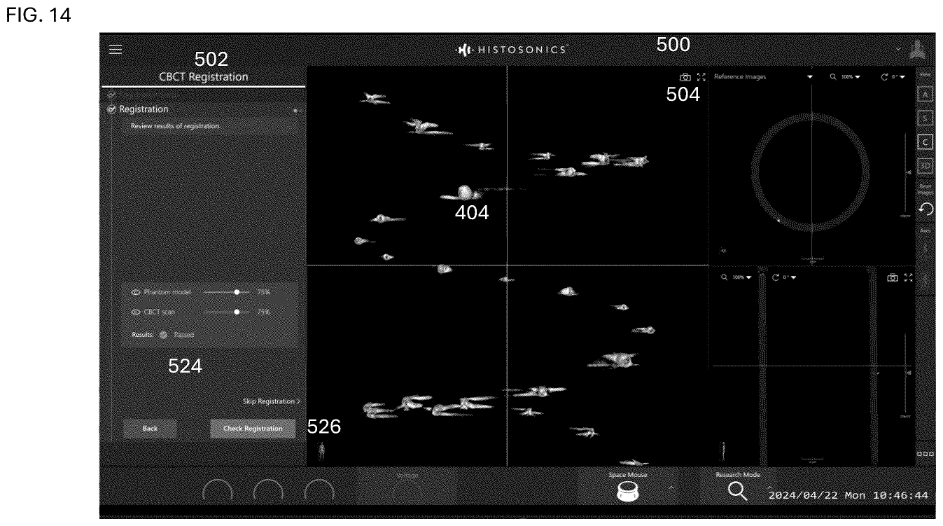

At FIG. 12, the instruction panel 502 provides instructions to utilize the cone-beam CT imaging device to acquire cone-beam CT images at step 616. Examples of both an acceptable image (e.g., good scan) and unacceptable images (e.g., poor scan) may be displayed respective portion of the image panel 504. This allows the user to view the captured images on a display screen connected to the cone-beam CT imaging device to determine whether the scan was a “good scan” at step 618. In some example configurations, users may be guided to use standard scan parameters. In other configurations, the cone-beam CT imaging device may be configured with specific scan parameters or protocols based on use case or application (e.g., liver/abdominal versus neuro/spine, etc.). If not, the back button 516 may be selected and the method returns to step 610 and the position of the phantom 402 can be adjusted. If yes, then the import CT scan button 518 may be selected and the workflow progresses to FIG. 13 where a file director 520 allows for selection of a cone-beam CT image file 521. Once highlighted via user input, the import button 522 may be selected and the highlighted cone-beam CT images are imported into the histotripsy system 100 at step 620 and the workflow progresses to the user interface 500 depicted in FIG. 14.

Various means and methods may be used for transferring the image data from the cone-beam CT imaging system to the histotripsy system 100. In some embodiments, a network integration may exist between the cone-beam CT imaging device and the histotripsy system 100, wherein the histotripsy system 100 receives and displays the registration scan without requiring the user to search directories for it, including embodiments where this is enabled to automatically transfer and load the most recent registration scan. In other embodiments, there may be further watchdogs that require updated or re-verified registrations based on time thresholds or detected movement of one or more systems.

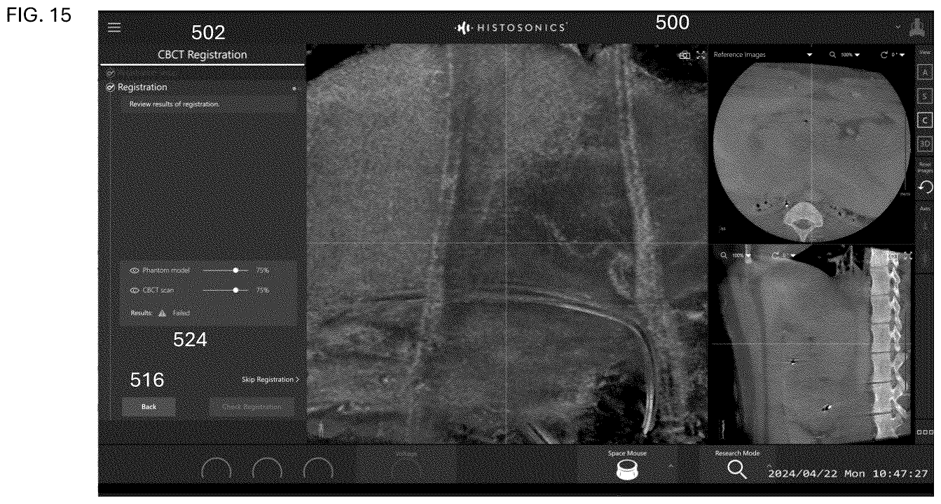

In FIG. 14 the cone-beam CT images are displayed in image panel 504. As can be seen in FIG. 14, the markers 404 of the phantom 402, which are the only portions of the phantom 402 which are readily observable in the cone-beam CT images. The position and orientation of the markers 404 in the images is compared using image processing to confirm whether the detection of the position, orientation, and size of the markers 404 in the images corresponds to their known relative positions, orientations, and size in the phantom 402. In addition, a determination is whether a sufficient number of markers 404 is detected in the images. When a determination is made that the cone-beam CT images are acceptable (yes at step 622) an indicator 524 is depicted in the instruction panel 502, and the check registration button 526 may be selected. However, if the images are unacceptable (no at step 622), as depicted in FIG. 15, where the incorrect image file was selected in FIG. 13, the indicator 524 indicates that the images are unacceptable. If the wrong image file was selected, selection of the back button 516 returns the method to FIG. 13 and step 620 where a different image file may be selected. Alternatively, the method may return to step 510 and the position of the phantom may be adjusted and the method repeats from that point until captured images from the cone-beam CT imaging device are found acceptable.

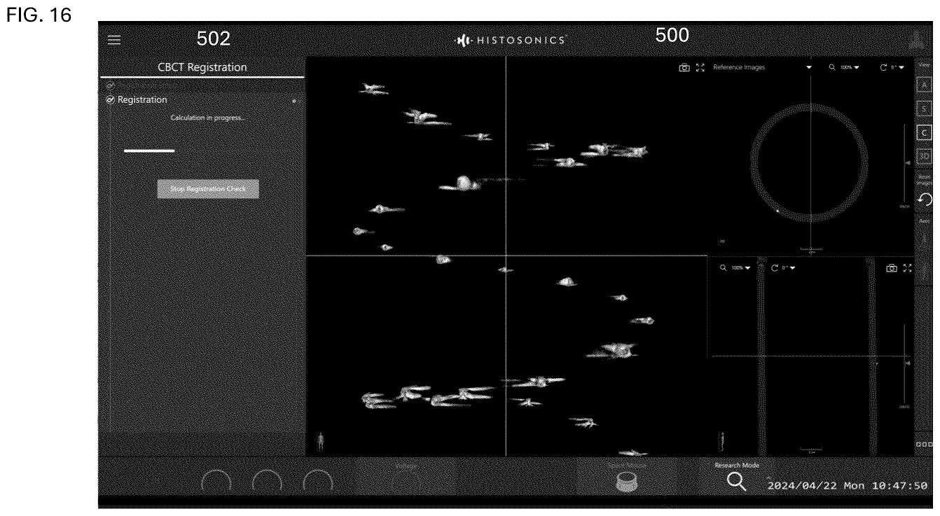

As the user proceeds through the guided work-flow, they may be prompted by the check registration button 526 to executes further steps to check or verify the registering of the known position of the markers 404 in the phantom 402 in the coordinate system of the robotic positioning arm 108 to the detected position of the markers in the cone-beam CT images at step 624. An indicator 528 in the instruction panel 502 displays the process of the registration calculation, as shown in FIG. 16.

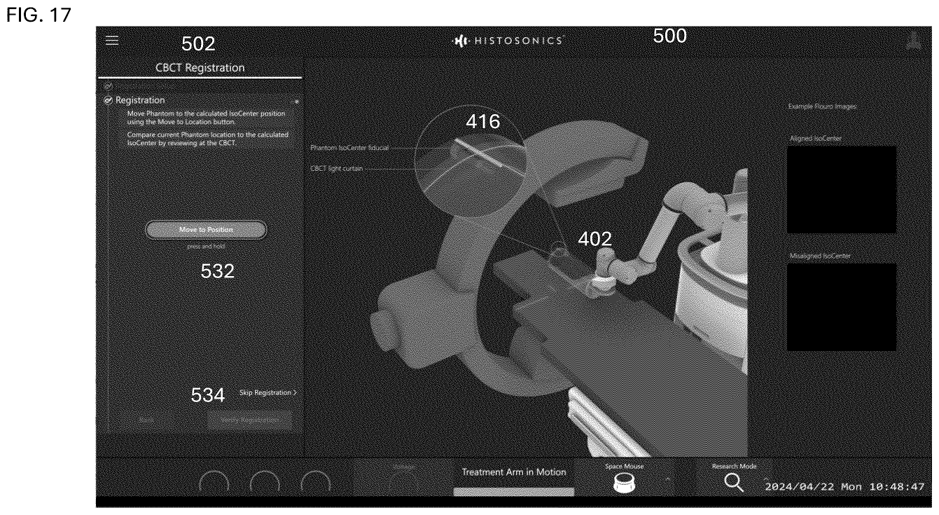

Once the registration calculation is complete the method moves to user interface 500 as depicted in FIG. 17, where a registration confirmation process is undertaken. At step 626 confirmation is undertaken to confirm that the robotic positioning arm 108, whose coordinate system is now registered to the cone-beam CT imaging coordinates can be accurately driven to a known location in the cone-beam CT imaging device coordinate system (e.g., the iso-center of the cone-beam CT imaging device). As shown in FIG. 17, the instruction panel 502 includes instructions to move the phantom 402 so that an iso-center alignment marker 416 is aligned with the iso-center of the cone-beam CT imaging device. The iso-center alignment marker 416 is a visible maker located at a known location and orientation of the phantom 402. When the light emitters of the cone-beam CT imaging device are toggled on to identify the iso-center of the cone-beam CT imaging device, the iso-center alignment marker 416 should axially align with one line of light formed by the light emitters, and a second perpendicular line of light formed by the light emitter should bisect the iso-center alignment marker 416, as depicted in the magnified view 530 in image panel 504. As previously described, if light emitters are not available on a given cone-beam CT system, the isocenter position may be checked using fluoroscopy from orthogonal imaging planes. The phantom 402 is automatically moved to the cone-beam CT imaging device iso-center by selecting and holding the move to position button 532 in the instruction panel 502. At step 628 a determination is made whether the iso-center marker is positioned at the cone beam CT imaging device iso-center. Once the iso-center alignment marker 416 is confirmed at the iso-center of the cone-beam CT imaging device, the verify registration button 534 can be selected at step 630, and the registration method registering the coordinate system of the cone-beam CT imaging device to the coordinate system of the histotripsy system and particularly the robotic positioning arm 108 is complete. At step 628 if the iso-center alignment marker 416 is not at the iso-center of the cone-beam CT imaging device the method may return to step 608 and new images acquired for registration and the registration method can be repeated.

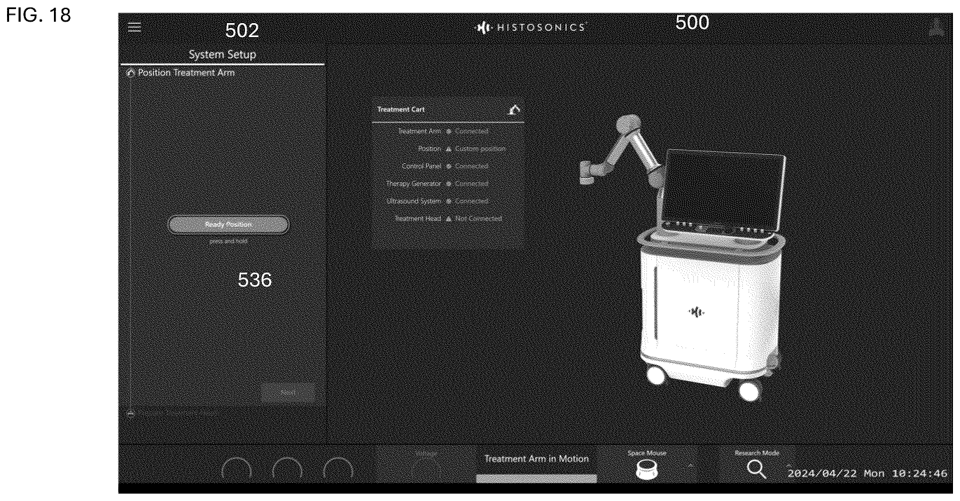

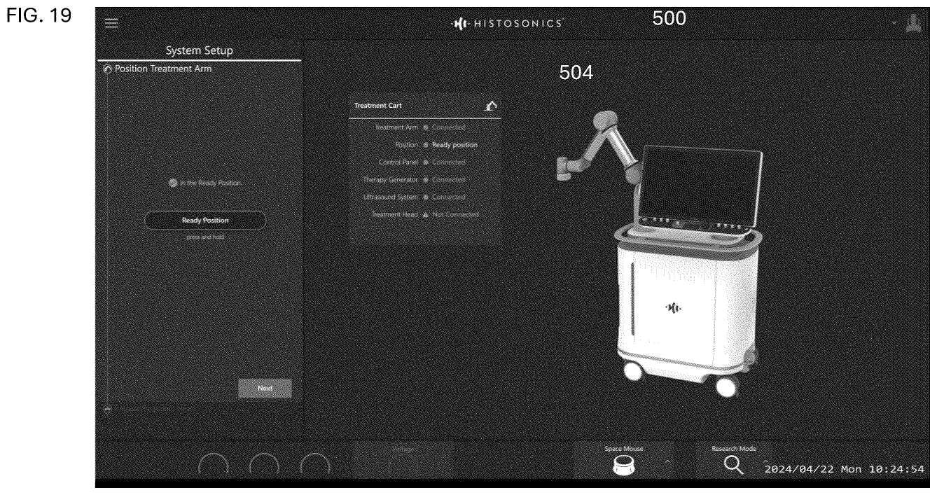

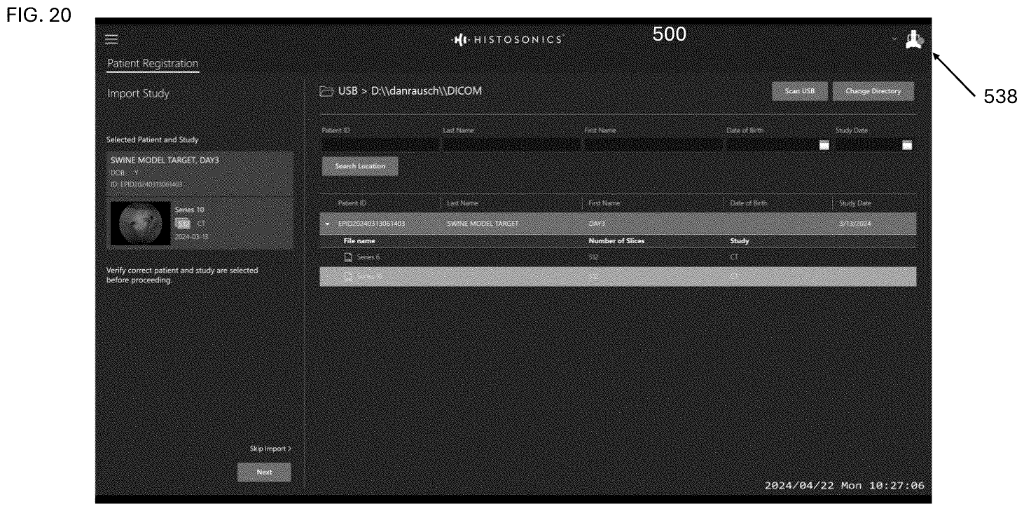

Following verification of registration at step 630 by selection of the verify registration button 534, the method progresses to FIG. 18 where the ready position button 536 can be selected. Selection of the ready position button 536 moves the robotic positioning arm 108 to a location where the phantom 402 can be removed. FIG. 19 displays an indicator in the instruction panel 502 that the robotic positioning arm 108 has achieved the ready position at which the phantom 402 can be removed and the treatment head (e.g., therapy transducer 102) can be connected. Once the treatment head is connected, the image panel will display a “connected indicator” next to the image of the histotripsy system 100 and a separate indicator (e.g., green check mark on the treatment head symbol 538) will display throughout the procedure in the user interface 500 (see e.g., FIG. 20).

Once the cone-beam CT imaging device coordinate system and the histotripsy system 100 coordinate system are registered to one another, so long as the cart 210 of the histotripsy system 100 and iso-center of the cone-beam CT imaging device are not moved the coordinate system of the cone-beam CT imaging device and the coordinate system of the robotic positioning arm 108 are transformable from one to the other and either can be employed to provide guidance to either or both pieces of equipment. As noted above, at step 614 the wheels of both systems are locked during the registration method 600. In one embodiment of the disclosure, both the histotripsy system 100 and the cone-beam CT imaging device may include one or more accelerometers, gyroscopes, or other motion detectors configured to detect movement or shocks that could negatively affect the registration. An indicator may be presented on the user interface 500 in the display and control panel 106 when such movement or shock is detected.

In accordance with a further aspect of the disclosure, an optical tracking system may be employed. The optical tracking system allows for registration of the cone-beam CT imaging device to the histotripsy system 100 to be updated if either or both are ever moved within the surgical room after registration. In accordance with this aspect of the disclosure an optical tracking system is employed. The optical tracking system may employ one or more infrared cameras and a plurality of optical markers. The optical markers may be active (light emitters) or passive (light reflectors). The optical markers may be placed on equipment such as the cone-beam CT imaging device, the histotripsy system 100, and in accordance with one aspect of the disclosure on the phantom 402. Each optical tracker is located on a portion of the equipment which itself does not typically move during a procedure (e.g., on the cart 210, or a non-moving portion of the cone-beam CT imaging device) and not on a moving element such as the C-arm of the cone-beam CT imaging device or on the robotic positioning arm 108). In accordance with the disclosure following step 628 by selection of the verify registration button 534, rather than move to a position to connect the therapy transducer 102, the application may display in the UI an indicator to confirm optical scanning engaged and all markers are detected. This may optically include the optical trackers on the phantom 402 positioned at the iso-center of the cone-beam CT imaging device. This provides a relative location of the iso-center of the con-beam CT imaging device to the marker(s) located on the cone-beam CT imaging device. Once all markers are detected, their relative locations determined, and the registration method confirmed complete, the optical tracking system can supply data to the histotripsy system 100 to update the registration to account for any detected movement of the cone-beam CT imager or histotripsy system 100 relative to each other. In this way, the registration process for the cone-beam CT imaging device need not be repeated with each procedure. The application may additionally display an indicator that re-registration is required if the relative movement of the equipment becomes too great, if the optical tracking system is unable to detect one or the markers, or on a periodic basis (e.g., quarterly or yearly).

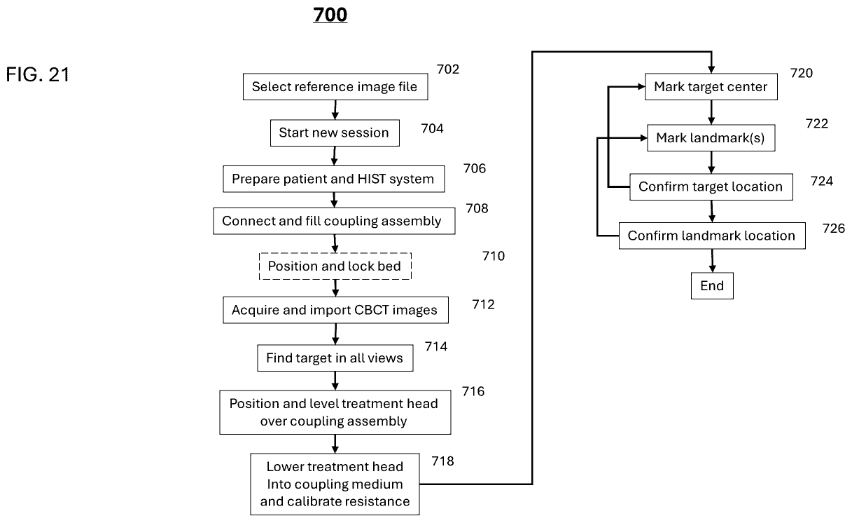

At any point following registration of the cone-beam CT imaging device to the histotripsy system 100, a histotripsy procedure utilizing cone-beam CT images of the patient may be undertaken. FIG. 20 depicts an initial user interface 500 of a histotripsy method 700 outlined in FIG. 21 for performing a histotripsy procedure employing cone-beam CT images acquired intraprocedurally.

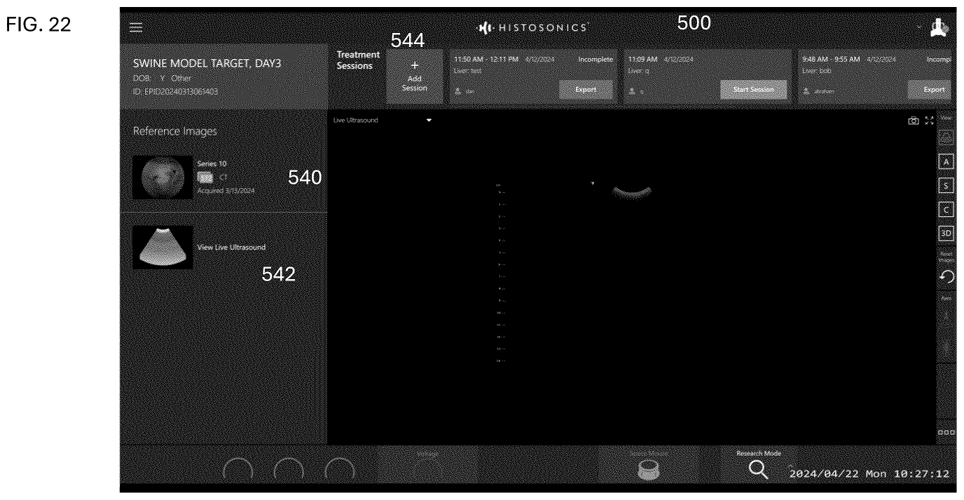

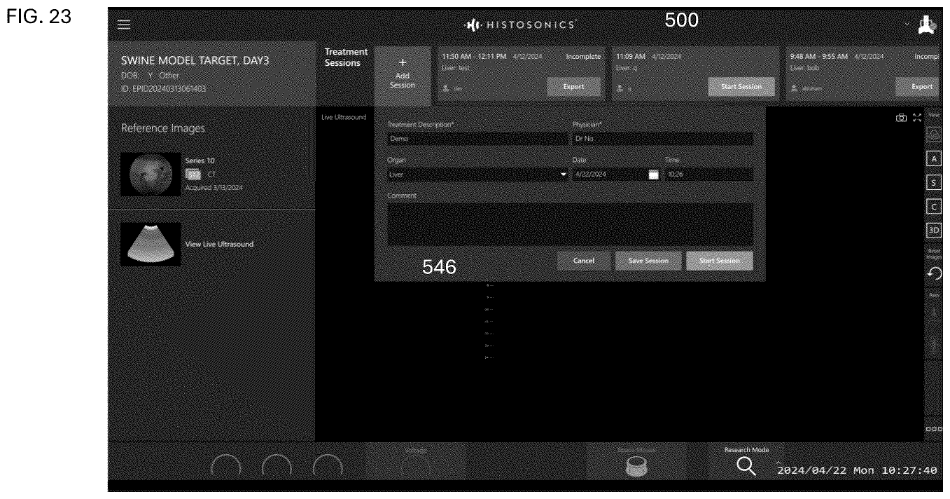

At step 702, and as shown in FIG. 21, a reference image file is selected via the user interface 500. The reference image file may be MR images, CT images, cone-beam CT images, PET images, or other relevant forms of images useable to diagnose and identify target tissue (e.g., a tumor or lesion) within the patient. With the image file selected, the user interface 500 transitions to FIG. 22, where an indicator 540 of the reference image file is displayed along with an indicator to view live ultrasound images 542. An add session button 544 can be selected and a drop-down menu 546 (FIG. 23) is displayed in the user interface 500. Because the selected reference image file includes personal data of the patient. This data can be read from the reference image file and auto populates the fields of the drop-down menu 546. Any missing data can be added and once complete at step 704 a start session button 549 can be selected.

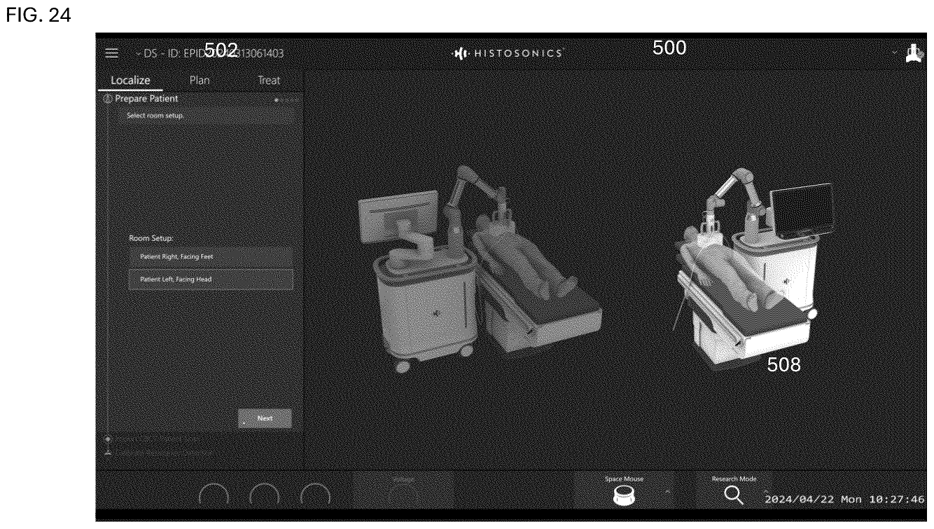

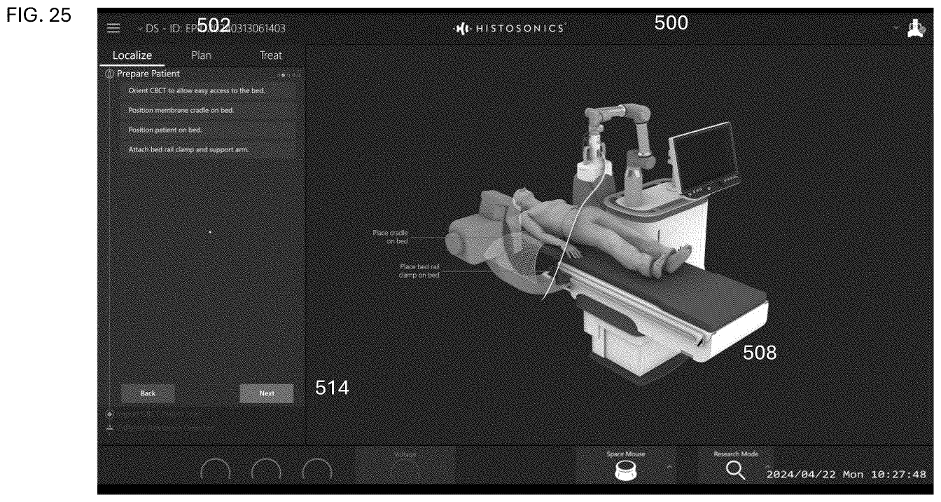

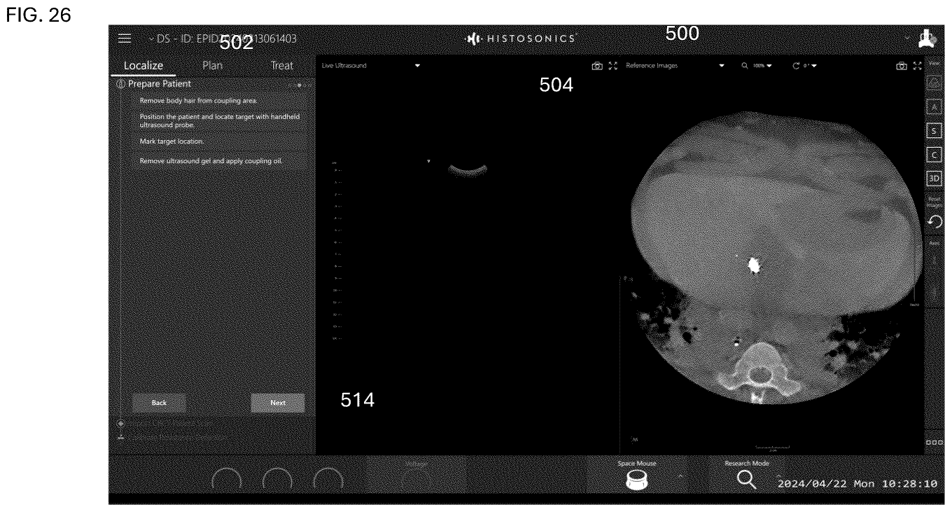

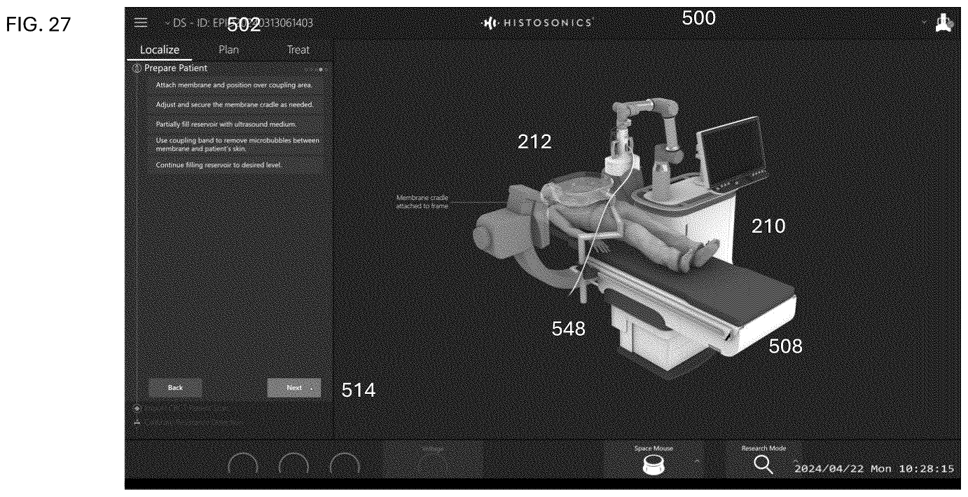

The method 700 then advances the user interface 500 to the view depicted in FIG. 24. The instruction panel 502 in FIG. 24 depicts three tabs, each tab representing a phase of the procedure. The first phase is the localize phase, the subsequent phases include plan and treat phases. Aspects of this disclosure focus primarily on the features of the localize phase when utilizing cone-beam CT images. A first step in the localize phase requires a user to select an orientation of the histotripsy system 100 with respect to the bed 508 at step 706. Once the orientation of the histotripsy system 100 is selected, the method advances to the user interface 500 depicted in FIG. 25. In FIGS. 25-28 the instruction panel 502 presents instructions for the surgical staff to perform in preparing the histotripsy system 100 and the patient for the procedure including re-orienting the cone-beam CT imaging device to allow access to the bed 508 and placing the membrane constraint 216 on the bed 508. This may specifically include adjusting the angulation of the cone-beam CT to allow better access to the bed (e.g., lateral imaging position) while maintaining the registration accuracy. Once so arranged the patient can be placed on the bed 508 and a bed rail 548 (FIG. 27) that will support the coupling assembly 212 can be attached to the bed 508. After selecting the next button 514 further instructions are presented in the instruction panel in FIG. 26 including instructions to remove body hair from the patient in the coupling area and using handheld ultrasound to locate the target tissue in the patient. Further there are instructions to mark the location on the patient at which the target can be viewed. Finally, the ultrasound gel is to be removed and the coupling oil applied to the patient. Again, following some or all of these steps the next button 514 can be selected.

As noted above, one of the features of the integration of the histotripsy system 100 with the cone-beam CT imaging device is that it enables application of therapy even where the target tissue cannot be observed in ultrasound imaging (e.g., where blocked by dense tissue such as bones). Thus, not all of the instructions need be completed to select the next button 514. In connection with FIG. 26, the ultrasound images may be displayed in the imaging panel 504 either by themselves or in combination with the reference images.

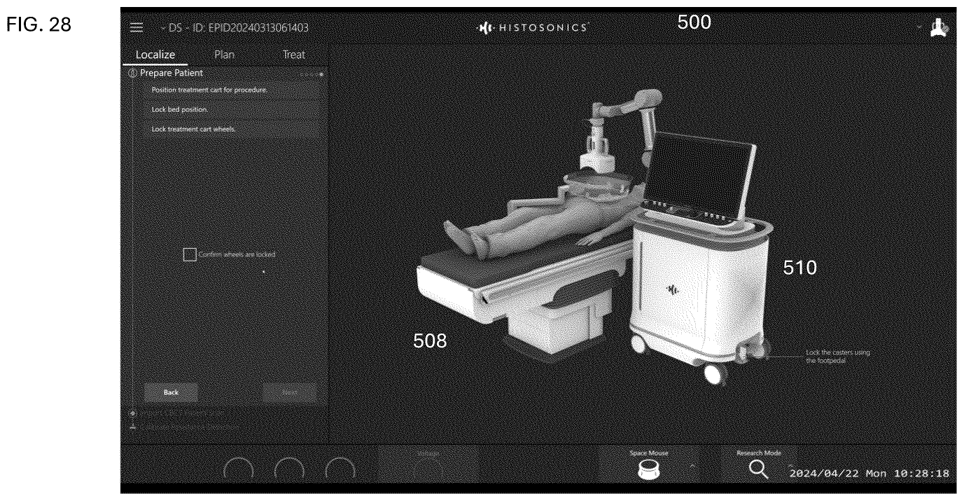

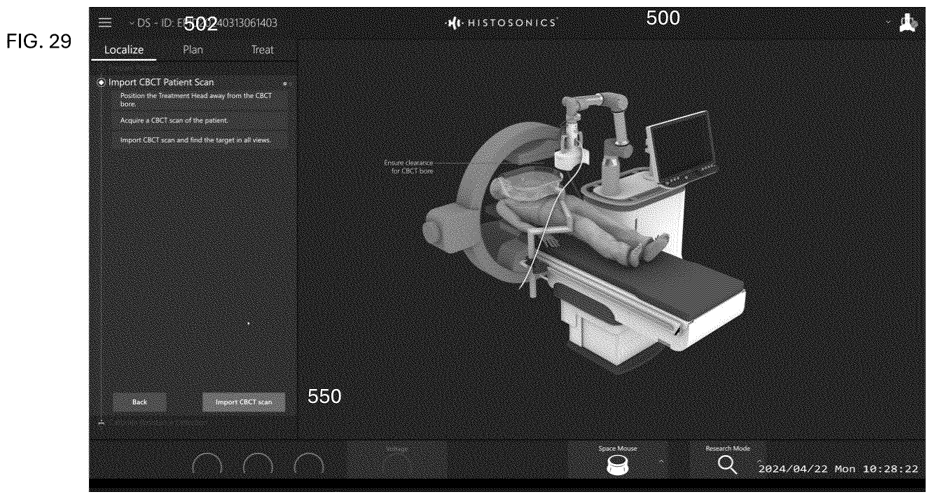

1. Once the next button 514 is selected in FIG. 26 the method advances to FIG. 27 where the instruction panel 502 provides additional instructions for step 708 regarding attaching the coupling assembly 212 to the bed rail 548 and filling the coupling assembly 212. Once the coupling assembly 212 is attached to the bed rail 548 and filled with the ultrasound medium (e.g., degassed water) the next button 514 may be depressed advancing the method to FIG. 28. As shown in FIG. 28 the instruction panel 502 provides for step 710 for positioning and locking of the wheels of the bed 508. As will be appreciated, when employing the cone-beam CT imaging device, this positioning and locking of cart 210, and the cone-beam CT imaging device should already have taken place during the registration process (above). Once an input is received confirming the final positioning and locking of the bed 508, the next button 514 will illuminate and can be selected, advancing the method to user interface 500 as depicted in FIG. 29. In some cases, the bed will have to be adjusted from the registration steps in order to properly position and iso-center the patient with the cone-beam CT. Once the patient anatomy is iso-centered and clearance is checked (for collisions), the bed may then be locked for the remainder of the procedure to ensure accurate patient registration during the subsequent procedure steps.
2. In FIG. 29, the instruction panel 502 presents instructions for acquiring a cone-beam CT scan of the patient. In accordance with the disclosure, operation of the cone-beam CT imaging device is separate from the control and operation of the histotripsy system 100, however, the disclosure is not so limited and it is contemplated that a user may operate the cone-beam CT imaging device via the user interface 500 on the display and control panel 106. The instructions displayed include directing the user to position the therapy transducer 102 away from the cone-beam CT imaging device. Further in accordance with step 712 the instructions direct the surgical staff to acquire cone-beam CT images utilizing the cone-beam CT imaging device. As with the registration step, the surgical staff is able to review these images in a display associated with the cone-beam CT imaging device, and when acceptable can select the import CBCT scan button 550. As previously described, various means of transferring images and corresponding data may be utilized, including but not limited to direct network connections.

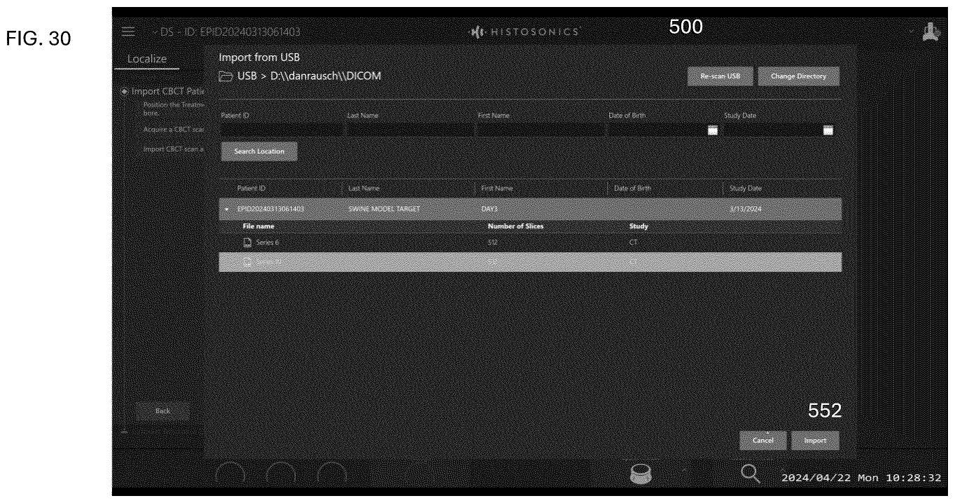

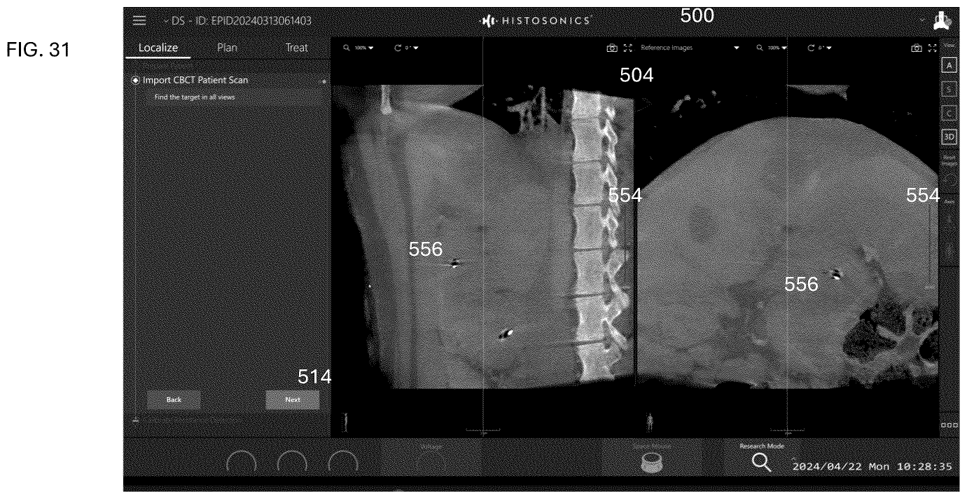

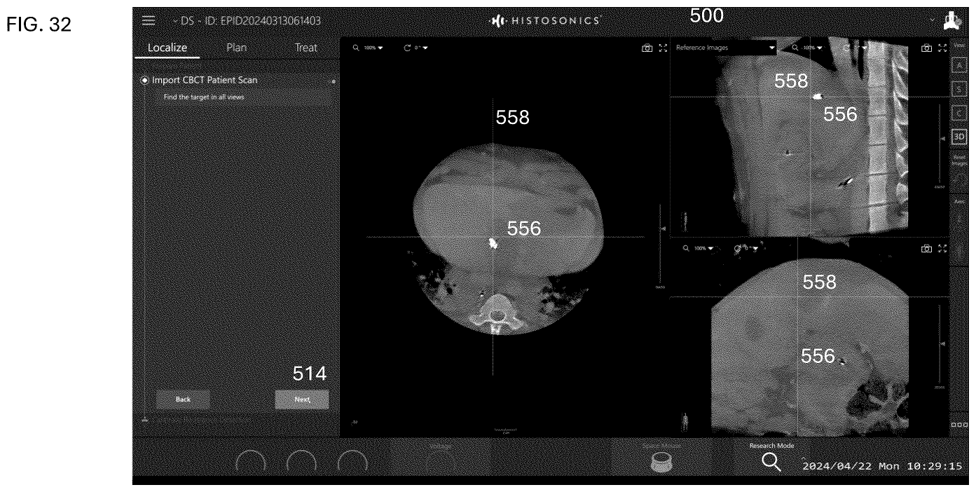

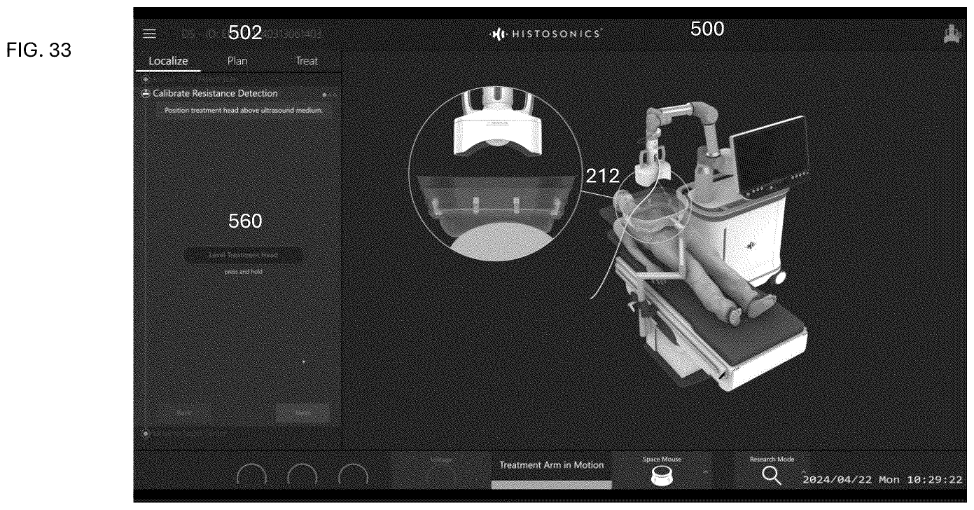

Once import CBCT scan button 550 is selected, the method progresses to user interface 500 depicted in FIG. 30 where a cone-beam CT image file may be selected from the list. Once selected and the import button 552 is selected, and the select image file is imported into the histotripsy system 100. As will be appreciated, in some aspects of the disclosure, no selection is necessary and after acquisition the cone-beam CT image file (e.g., a CBCT DICOM file) can be automatically transferred to the histotripsy system 100 following acquisition by the cone-beam CT imaging device. The method 700 moves to FIGS. 31 and 32, which depict in the image panel 504 various views (e.g., coronal, sagittal, axial, etc.) of the images acquired and imported at step 712. With the images displayed, scroll tabs 554 can be manipulated to scroll through the images and identify the target 556 in all views. The cross hairs 558 are placed on or near the target in each view. Once achieved, the next button 514 is selected and the method 700 progresses to user interface 500 as depicted in FIG. 33.

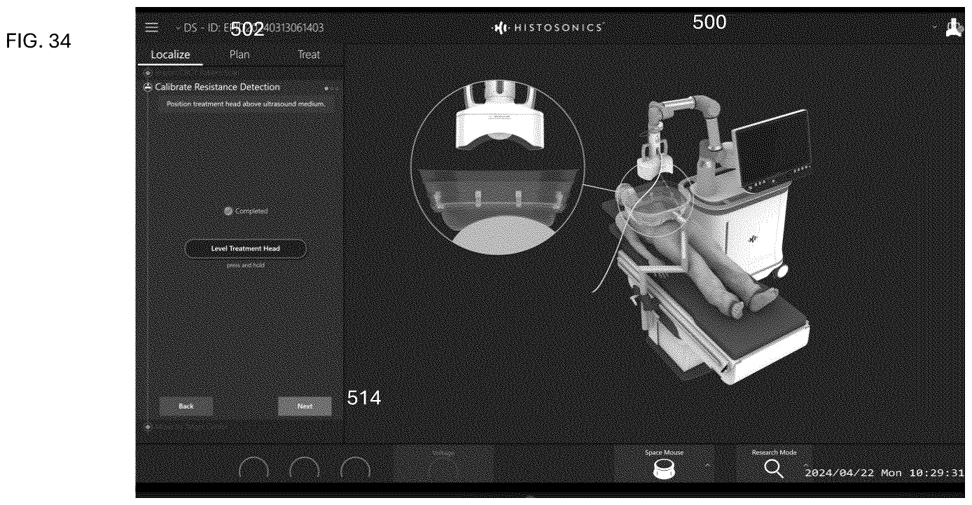

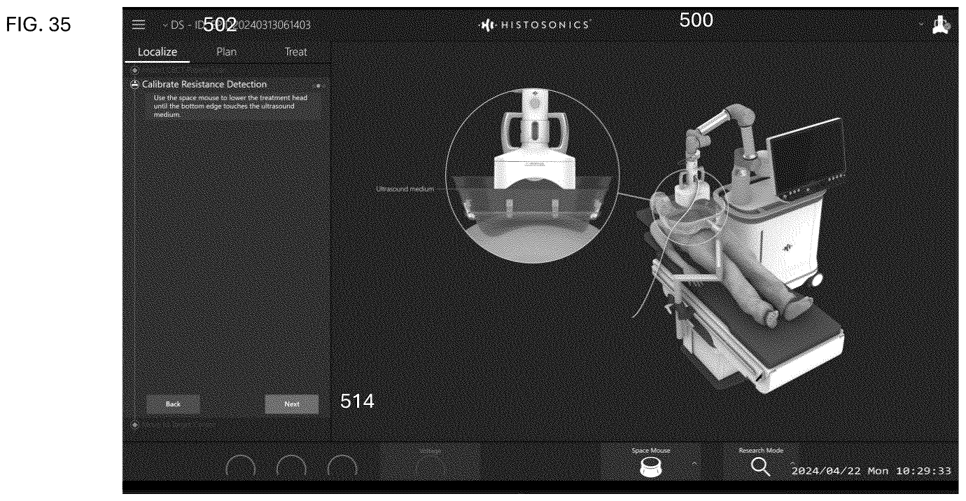

In FIG. 33 the instruction panel 502 in connection with step 716 directs the surgical staff to position the therapy transducer 102 over the coupling assembly 212. Once so positioned, as depicted in FIG. 34 the level treatment head button 560 may be selected leveling the orientation of the therapy transducer 102 relative to the coupling medium in the coupling assembly 212. Once leveled and an indicator of completion is displayed the next button 514 may be selected advancing the method 700 to the user interface 500 depicted in FIG. 35.

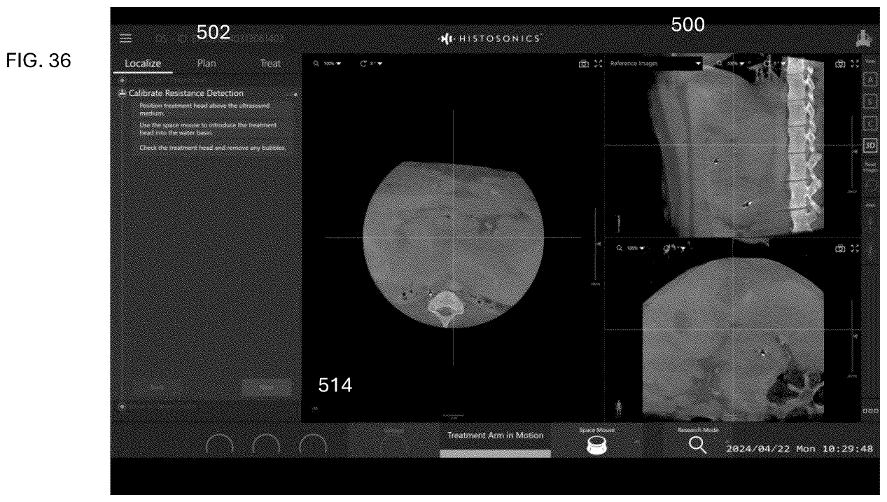

At step 718, and as instructed in the instruction panel 502 in FIGS. 35 and 36 the treatment transducer 102 is lowered into the coupling assembly 212 and coupling medium contained therein. Any bubbles under the therapy transducer 102 should be removed. Once all bubbles are removed and the treatment transducer is submerged the next button 514 in FIG. 36 will illuminate and can be selected.

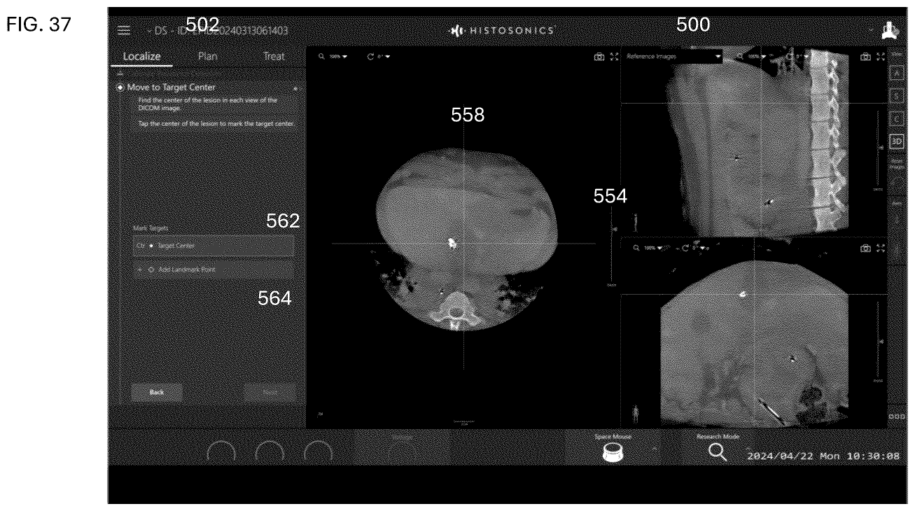

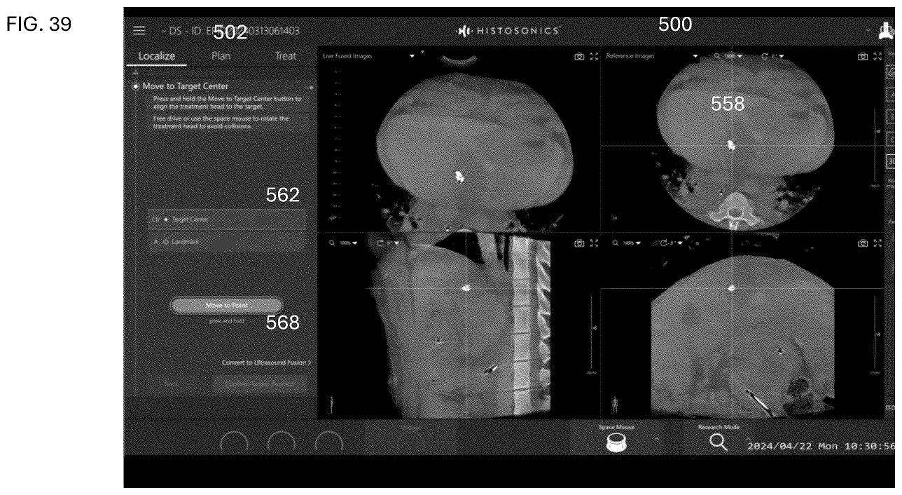

Selection of the next button 514 advances the method to FIG. 37 where the instruction panel 502 asks the surgical staff to mark the center of the target tissue in all views of the imported cone-beam CT images (e.g., coronal, sagittal, axial) displayed in the image panel 504. A target center button 562 is selected, and the surgical staff uses the scroll tabs 554 to scroll through the cone-beam CT images then place the crosshairs 558 at the center of the target tissue (e.g., a tumor or lesion) in all three views at step 720. Once the crosshairs 558 are at the center of the target tissue, at step 722 the landmark button 564 is selected and again the images are scrolled through using the scroll tabs 554 to identify a landmark (e.g., blood vessel, rib, or other structure) that is visible in all three views. In some examples, landmarks are ideally selected that are in close proximity to the target and that may be visible on both cone-beam CT and ultrasound views. A second landmark button 566 is displayed in FIG. 39 and may be selected to identify a second landmark identified using the same process. Once at least the target is identified in the cone-beam CT images, the next button 514 may be selected, the locations of the target tissue center and any optional landmarks are saved in the memory of the histotripsy system.

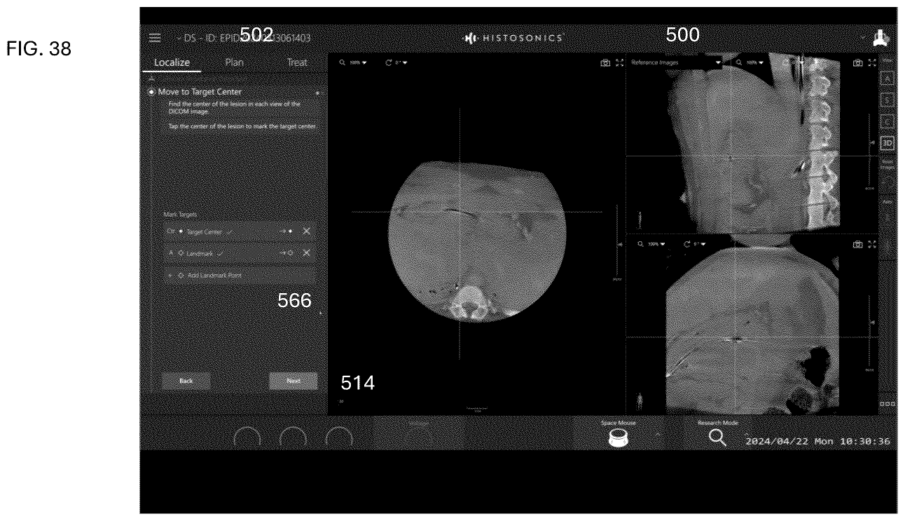

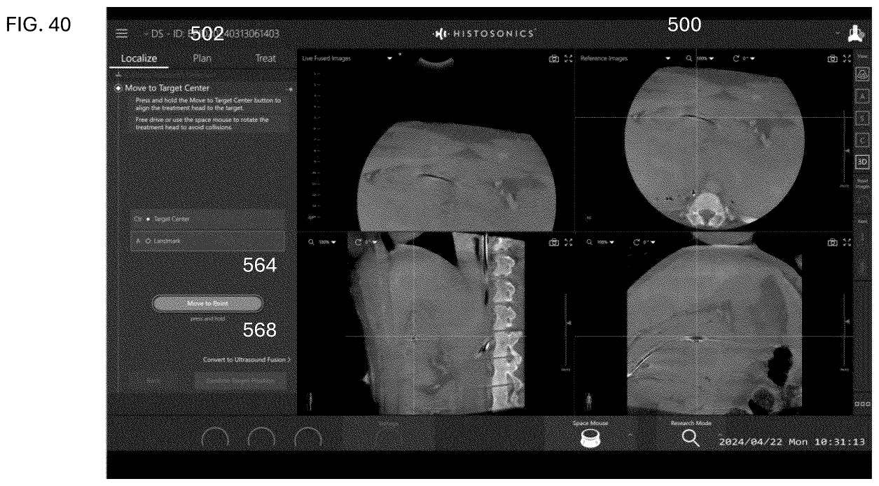

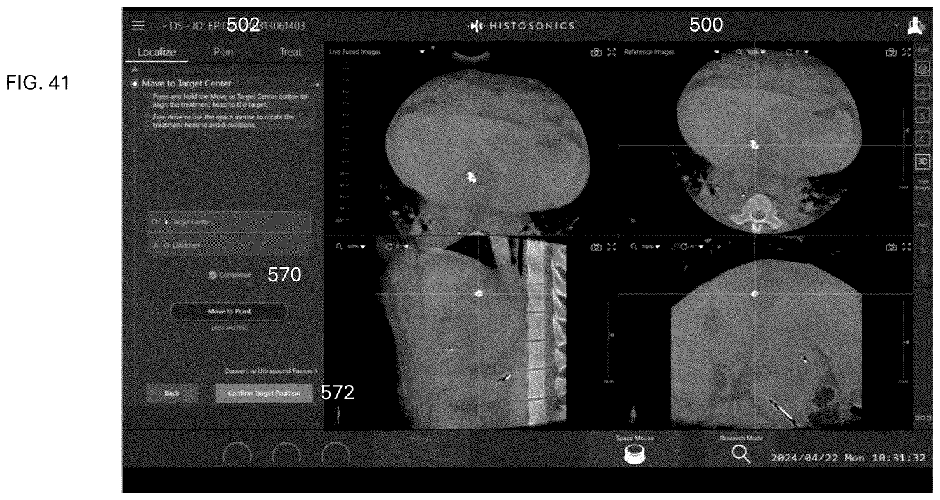

By selecting the next button 514 the method advances to FIG. 39. In FIG. 39 by selecting the target center button 562, and then depressing and holding the move to point button 568 the cone-beam CT images are automatically scrolled through to display the target tissue and the crosshairs 558 at the center of the target tissue to confirm identification of the center of the target tissue at step 724. This scrolling of the cone-beam CT images coincides with the movement of the robotic positioning arm 108 to a location at which the focal point of the therapy transducer 102 is centered on the target tissue. Similarly, by selection of the landmark button 564 the method advances to FIG. 40, and again the move to point button 568 is held and the cone-beam CT images are automatically scrolled through to display the previously marked location of the landmark (FIG. 38). Again, this coincides with the movement of the robotic positioning arm 108 to a location at which the focal point of the therapy transducer is centered on the landmark. Once both the target center and the landmark have been automatically scrolled through and visually confirmed, and the movement of the robotic positioning arm 108 has been achieved without any interference from the patient, the CBCT imaging device, or any other component an indicator of completion 570 is displayed in the user interface 500 in FIG. 41. The user may then select that confirm target position button 572 button and the localize phase is complete. During any of steps 724 or 726 a back button may be selected returning the method 700 to step 720 or step 722 respectively. During the movement of the robotic positioning arm 108 a distance of the location of the focal point to the target center or landmark may be displayed and updated in the user interface 501 as the focal point approaches the target tissue. Described in greater detail below in connection with 42-46, the user interface 500 of FIGS. 39 and 40 may also display cone-beam CT images fused with pre-procedure CT or live ultrasound images.

Because the coordinate systems of the cone-beam CT imaging device and the histotripsy system 100 have been registered, identification of the target tissue and/or a landmark in the cone-beam CT images provides a location to which the robotic positioning arm 108 and therapy transducer 102 can be navigated to for the application of therapy to the target tissue, unlike prior systems that require ultrasound imaging confirmation, this movement and application of therapy can be achieved relying just on the location data derived from the cone-beam CT images.

The planning and treatment phases of this disclosure are substantially the same as described in connection with commonly assigned and co-pending U.S. patent application Ser. No. 18/642,529, filed on Apr. 22, 2024 titled HISTOTRIPSY SYSTEMS AND ASSOCIATED METHODS INCLUDING USER INTERFACES AND WORKFLOWS FOR TREATMENT PLANNING AND THERAPY, the entire contents of which is incorporated herein for all aspects of the planning of therapy and the application of treatment using the user interfaces 500 and the corresponding user interfaces described therein. In the interest of brevity, unique aspects of the planning phase are described in greater detail below.

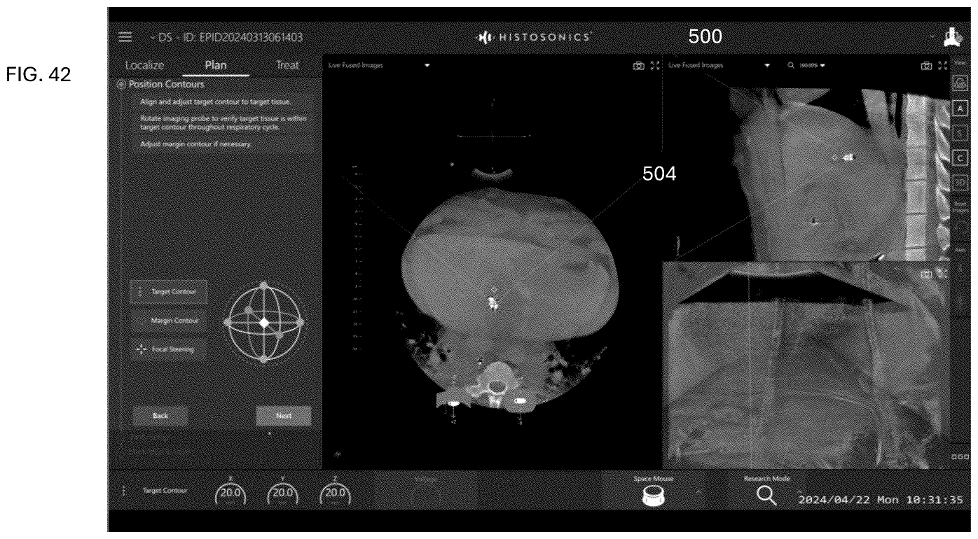

Though image fusion is an aspect of the prior application, the images fused in that application are pre-procedure CT or MR images fused with live ultrasound. In contrast, the live fused images depicted in FIGS. 42-46 are of the cone-beam CT images acquired in the localize phase. These can be fused with either live ultrasound or pre-procedure CT or MR images. Further, the relative opacity of images being fused together can be adjusted so that one is more prominent while the other is presented as a semi-transparent overlay.

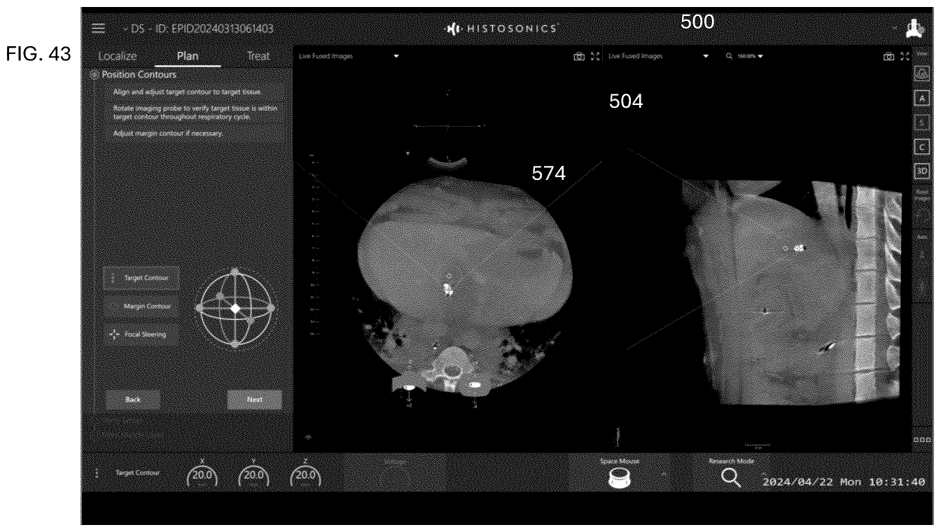

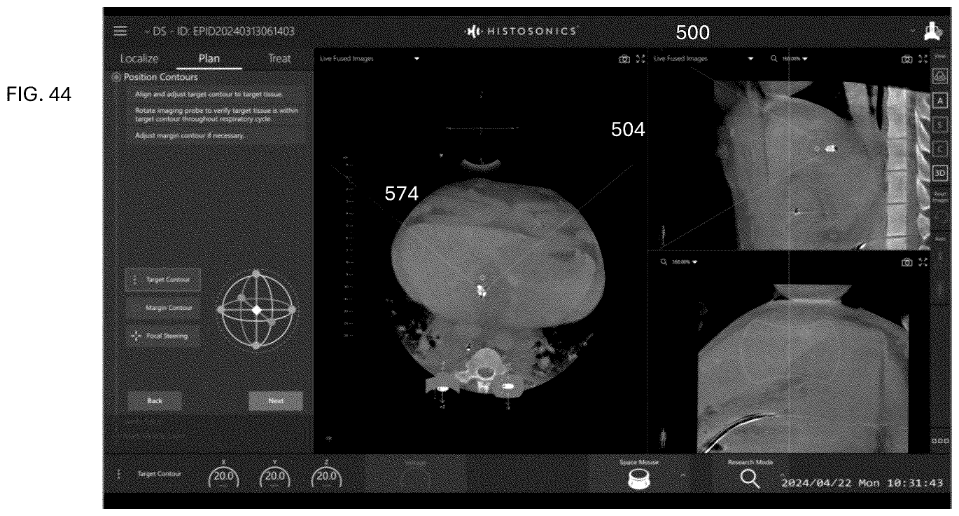

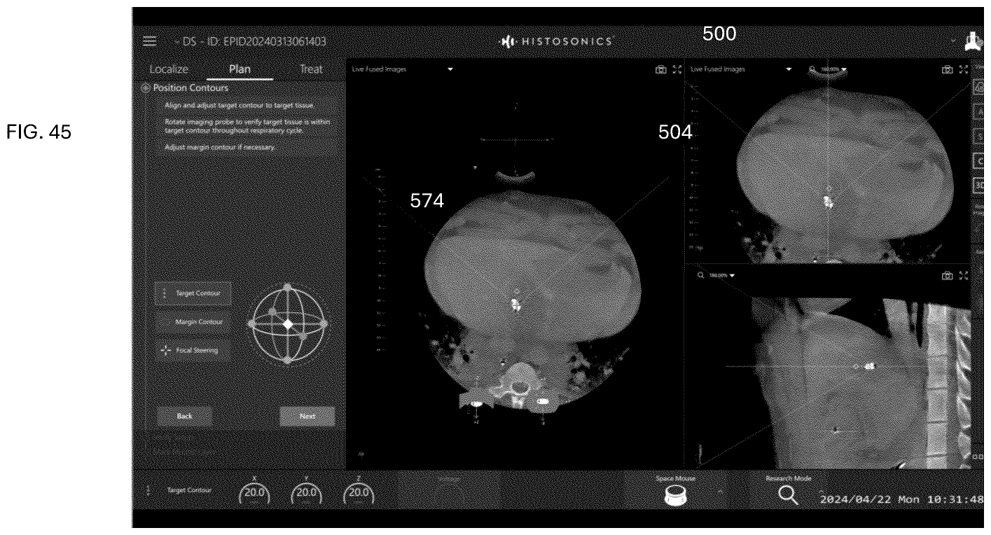

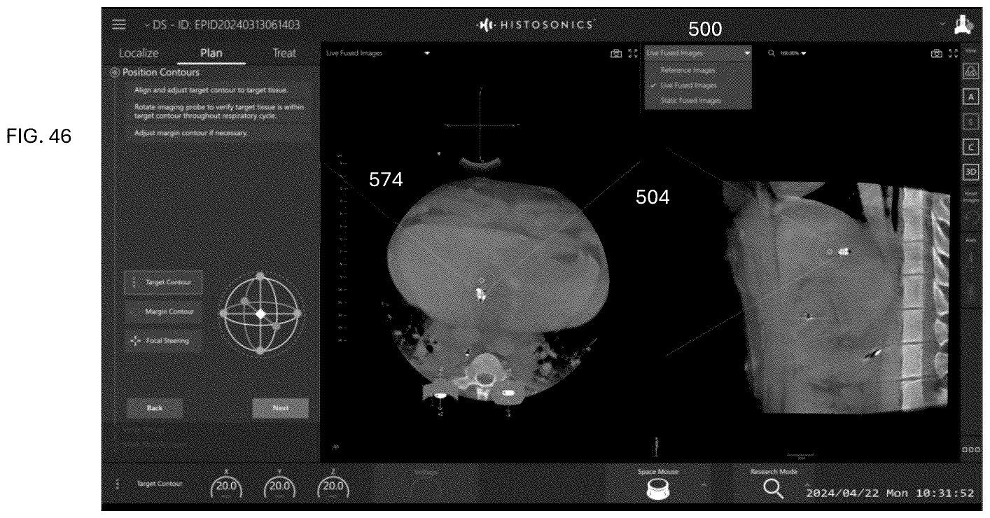

The images selected for display in the image panel 504 can be selected by the user. For example, in FIG. 42 along with an axial view, a sagittal view and a 3D view are presented in the user interface 500. In contrast, in FIG. 43 just the sagittal view is displayed alongside the axial view, in FIG. 44 just sagittal and coronal views are displayed, and in FIG. 45 the axial view is actually displayed twice along with the sagittal view. FIG. 46 depicts the dropdown 573 which is accessible in all views of the image panel 504 to select the type of image being displayed (e.g., reference or pre-procedure images, live fused images, or static fused images). As will be appreciated, live fused images are primarily useful when ultrasound imaging (e.g., with ultrasound imager 104) is available and useful for planning the therapy.

As described in the U.S. application Ser. No. 18/642,529, the planning involves identification of a target center and a plurality of end points orthogonal axes that traverse the target center. Of these points, a deepest point within the patient has importance to ensure that the focus of the therapy transducer 102 can reach this location without placing undue pressure on the patient. In some embodiments, this deepest location is the critical point to achieve. One indicator that the deepest point is achievable are the focal lines 574 depicted on the images in the image panel 504. Once all the end points are identified, a margin may be established around all of the end points, the margin is a portion of otherwise healthy tissue that is sacrificed to ensure that all of the target tissue (e.g., tumor or lesion) receives therapy. Finally, the focus of the therapy transducer 102 may be adjusted using focal steering to ensure that the margin, particularly at the deepest point within the patient can receive the therapy.

### Example

### Example 1: Cone-Beam CT Guided Liver Treatment

In one example, the disclosed concepts may be utilized to enable liver directed histotripsy therapy wherein one or more liver tumors may be located on the right side of the liver and with a corresponding poor acoustic window (e.g., intracostal only). The systems and methods disclosed herein are utilized to identify the one or more liver tumors and to localize/target them. In some related examples, intravenous contrast may be utilized to allow enhanced imaging of the liver.

### Example 2: Central Treatments in the Liver (Segment 1 or Caudate)

In one example, the configurations disclosed herein are utilized to enable treatment in central locations of the liver, of which are typically very challenging and/or not achievable due to the risk of injury to central vascular and/or biliary structures. The cone-beam CT configuration and method is utilized to localize and target the segment 1 and caudate lesions to ensure the planned treatment volumes are positioned in the optimal location in context to the structures.

### Example 3: Kidney Treatments Adjacent to the Ureter and/or Adjacent Bowel

In this example, the embodiments disclosed herein may be used to enable targeting small renal tumors that may be immediately adjacent to the ureter and surrounding bowel. The cone-beam CT based method is used, in conjunction with overlaid real-time ultrasound, to assess the planned treatment volume location and any interaction of the plan and the structures of interest. In one permutation of this example, the system and plan location may be adjusted to prevent overlap with the ureter and/or bowel. In another permutation, the system and plan location may be adjusted to allow plan interaction/overlap with the ureter and/or bowel, including a specific amount of overlap. In another example, the motion of the overlap may be studied with the real-time ultrasound and overlaid on the cone-beam CT images. The user may also be allowed to digitally reposition or modify the planned treatment volume location without moving the robot, and upon accepting the new position, the treatment head may move to the new user defined position and orientation.

### Example 4: Pancreas Directed Treatment for Treatment of Pancreatic Tumors

In this example, the user may use the disclosed systems and methods to target the head, body and/or tail of the pancreas. The user may utilize the features of the system to position the planned treatment volume in the desired plan location/orientation, and specifically in context to critical pancreas anatomical structures (e.g., pancreatic duct). The user may use the disclosed systems and methods to place a planned treatment volume that may intentionally be configured to not include treatment margin, and/or may be further configured to create a planned treatment volume that includes untreated locations within the tumor/lesion, in effort to minimize the damage to healthy, non-malignant, pancreas tissue. In this example, the user may utilize contrast enhance cone-beam CT images with real-time ultrasound overlays to assess the target location on both modalities, as well as the echogenicity of the target tumor tissue. In another permutation, the user may utilize various ultrasound imaging assessments (e.g., Doppler or elastography, etc.) to further assess the target and/or surrounding non-target tissue, as overlaid on the CBCT.

### Example 5: Bubble Cloud Calibration and Planning/Test Pulses

### Example 6

In one example, the systems and methods described herein for delivering cone-beam CT peri procedurally targeted treatments may further include steps that acquire cone-beam CT images during various phases of the workflow and ahead of “automated treatment”. This may comprise image acquisitions for assessing the spatial location (3D) of the artifact of the cloud (e.g., tissue effect) including the resulting tissue destruction of one or more bubble cloud treatment locations within the planned treatment volume (e.g., center point or plan extremes across X, Y, and/or Z coordinates). These steps may be further used to localize the actual resulting location of therapy delivery and following steps to calibrate/adjust in 2D, 3D and/or 4D based on the assessment on the acquired images. These steps may be utilized across the workflow to assist at one or more plan locations, during test or planning pulse steps and/or during automated treatment. As another example embodiment, the system may be configured to remember these locations/orientations and robot poses and allow the user to return to them as desired.

### Example 7. In Another Example and as Related to Example 6, the User May Also Use the Systems and Methods to Allow Immediate Post-Treatment Verification of the Treatment Plan

The embodiments described above are cited by way of example, and that the present invention is not limited to what has been particularly shown and described hereinabove. Rather, the scope of this disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are considered an integral part of the application.

We claim:

1. A system for registration, comprising:
a robotic arm operably connected to a histotripsy system;
a phantom operable connected to the robotic arm; and
a computing device operably connected to histotripsy system, the computing device including a processor and a memory, the memory storing therein instructions that when executed by the processor cause the computing device to perform steps of:
driving the robotic arm to an imaging location;
imports cone-beam computed tomography (CT) images of the phantom;
determines a number and orientation of markers within at least one of the imported cone-beam CT images; and
registers a coordinate system of the histotripsy system with a coordinate system of a cone-beam CT imaging device.

2. The system of claim 1, wherein the instructions when executed by the processor cause the computing device to display instruction via a user-interface to drive the phantom to an iso-center of the cone-beam CT imaging device.

3. The system of claim 2, wherein the instruction when executed by the processor cause the computing device to display instructions via a user interface to align a light alignment feature of the cone-beam CT imaging device with an iso-center alignment feature on the phantom.

4. The system of claim 1, wherein the instructions when executed by the processor cause the computing device to display instructions via a user interface to adjust a position of the phantom to a center of the cone-beam CT imaging device field of view, prior to capturing images of the phantom.

5. The system of claim 4, wherein the instructions when executed by the processor cause the computing device to display instructions via the user interface to align a light alignment feature of the cone-beam CT imaging device with one or more crosshairs on the phantom.

6. The system of claim 1, wherein the instructions when executed by the processor cause the computing device to display instructions via a user interface to attach the phantom to the robotic arm.

7. The system of claim 1, wherein the instructions when executed by the processor cause the computing device to display instructions via a user interface to arrange the cone-beam CT imaging device and the histotripsy system perpendicular to a patient bed.

8. The system of claim 7, wherein the instructions when executed by the processor cause the computing device to display instructions via the user interface to lock wheels of the cone-beam CT imaging device and the histotripsy system.

9. The system of claim 1, wherein the instructions when executed by the processor cause the computing device to display instructions via a user interface to confirm clearance of the cone-beam CT imaging device and the robotic arm.

10. The system of claim 1, wherein the instructions when executed by the processor cause the computing device to display instructions via a user interface to confirm a quality of the cone-beam CT images prior to importing the cone-beam CT images.

11. A method of registering a coordinate system of a cone-beam computed tomography (CT) imaging device and a robotic histotripsy system, comprising:
robotically driving a phantom connected to a robotic arm to an imaging location;
importing cone-beam CT images captured by a cone-beam CT imaging device of the phantom;
displaying an image from the imported cone-beam CT images of the phantom, wherein the displayed image depicts at least one marker in the phantom;
assessing a number and orientation of the at least one marker in the imported cone-beam CT images compared to a known number and orientation of the at least one marker in the phantom;
registering a coordinate system of the cone-beam CT imaging device to a coordinate system of the histotripsy system;
detecting a location of an optical marker on each of the phantom, the cone-beam CT imaging device and the histotripsy system; and
updating the registration of the cone-beam CT imaging device to the histotripsy system based on detected movement of the optical makers on the cone-beam CT imaging device and the histotripsy system.

12. The method of claim 11, further comprising:
driving the phantom to an iso-center of the cone-beam CT imaging device.

13. The method of claim 11, further comprising:
driving the phantom to a non-iso-center location of the cone-beam CT imaging device.

14. The method of claim 11, further comprising:
aligning a light alignment feature of the cone-beam CT imaging device with an iso-center alignment marker on the phantom.

15. The method of claim 11, further comprising:
adjusting a position of the phantom to a center of the cone-beam CT imaging device field of view, prior to capturing images of the phantom.

16. The method of claim 11, further comprising:
driving the phantom to a pre-determined location of the cone-beam CT imaging device.

17. The method of claim 16, wherein a light alignment feature of the cone-beam CT imaging device is aligned with one or more crosshairs on the phantom.

18. The method of claim 11, further comprising:
attaching the phantom to the robotic arm of the histotripsy system.

19. A registration phantom, comprising:
a body;
a plurality of markers secured to the body;
an angled attachment platform connected to the body; and
a connector configured for connection to a robotic arm.

20. The registration phantom of claim 19, wherein the plurality of markers is arranged in a helix around the body.

21. The registration phantom of claim 19, further comprising crosshairs formed on the body, wherein the crosshairs are used for alignment of the body with an imaging device.

22. The registration phantom of claim 21, wherein the imaging device is a cone-beam computed tomography system.

23. The registration phantom of claim 19, wherein the angled attachment platform is connected to the body at an angle selected to avoid robotic arm singularities.

24. The registration phantom of claim 19, further comprising an iso-center marker.

25. The registration phantom of claim 19, wherein the plurality of markers includes markers of two or more diameters.

26. The registration phantom of claim 19, further comprising one or more optical markers.

27. The registration phantom of claim 19, wherein the body is cylindrical.
